(19) United States
(12) Patent Application Publication
(10) Pub. No.: US 2020/0010854 A1
(43) Pub. Date: Jan. 9, 2020

(54) CRISPR/CAS9-BASED TREATMENTS

(71) Applicant: THE JOHNS HOPKINS UNIVERSITY, Baltimore, MD (US)

(72) Inventors: Albert S. JUN, Baltimore, MD (US); Vinod JASKULA-RANGA, Baltimore, MD (US); Donald ZACK, Baltimore, MD (US)

(21) Appl. No.: 15/741,444

(22) PCT Filed: Jul. 5, 2016
(86) PCT No.: PCT/US2016/040962

§ 371 Date: Jan. 2, 2018

Related U.S. Application Data
(60) Provisional application No. 62/188,013, filed on Jul. 2, 2015.

(51) Int. Cl. C12N 15/90; C12N 15/11

(52) U.S. Cl. CPC C12N 15/90; C12N 2320/30; C12N 2310/20; C12N 15/111

(57) ABSTRACT

Described herein are methods for treating disorders affecting ocular and non-ocular tissue, such as corneal dystrophies and microsatellite expansion diseases. The methods use a nuclease system, such as Clustered Regularly Interspaced Short Palindromic Repeats (CRISPR)/CRISPR associated (Cas) 9 (CRISPR-Cas9), to cut and/or repair genomic DNA. Such methods may further comprise a DNA double-stranded break (DSB) repair system comprising a repair template in combination with a Non-Homologous End-Joining (NHEJ) or Homology Directed Repair (HDR) targeted to the one or more CRISPR-Cas9 cleavage sites.

### CROSS REFERENCE TO RELATED APPLICATIONS

This application claims priority to U.S. Provisional Application No. 62/188,013, filed Jul. 2, 2015, the contents of which is incorporated herein by reference in its entirety.

### BACKGROUND

Corneal dystrophies are a group of disorders that are generally inherited, bilateral, symmetric, slowly progressive, and not predominantly related to environmental or systemic factors (1,2). Corneal dystrophies can affect any anatomic layer, cell type, or tissue of the cornea and result in loss of corneal clarity and reduction in vision (1,3). Corneal dystrophies as a group affect >4% of the US population, and corneal transplantation is definitive treatment for corneal dystrophies of sufficient severity to cause significant vision loss. Fuchs endothelial corneal dystrophy (FECD) is the most common corneal dystrophy affecting approximately 4% of the US population. Approximately 70% of FECD cases are caused by a microsatellite trinucleotide repeat expansion in the transcription factor 4 (TCF4) gene (4). Additional microsatellite expansion diseases have been described (5).

Thus there is a great need for novel and improved therapies for treating disorders affecting ocular and non-ocular tissues, like corneal dystrophies and microsatellite expansion diseases affecting the eye and other tissues and organs throughout the body.

### SUMMARY OF THE INVENTION

Described herein are methods for treating disorders affecting ocular and non-ocular tissues, such as corneal dystrophies and microsatellite expansion diseases. The methods use a nuclease system, such as Clustered Regularly Interspaced Short Palindromic Repeats (CRISPR)/CRISPR associated (Cas) 9 (CRISPR-Cas9), to cut and/or repair genomic DNA. The CRISPR-Cas9-based gene editing can be used to inactivate or correct gene mutations causing corneal dystrophies and microsatellite expansion diseases, thereby providing a gene therapy approach for these groups of diseases.

One aspect of the invention relates to a method for treating a disorder affecting ocular tissue in a subject, the method comprising administering to the ocular area of the subject a therapeutically effective amount of a nuclease system comprising a genome targeted nuclease and a guide DNA comprising at least one targeted genomic sequence.

In certain embodiments, the nuclease can be provided as a protein, RNA, DNA, or an expression vector comprising a nucleic acid that encodes the nuclease.

In certain embodiments, the guide DNA can be provided as an RNA molecule (gRNA), DNA molecule, or an expression vector comprising a nucleic acid that encodes the gRNA.

In certain embodiments, the guide DNA may be provided as one, two, three, four, five, six, seven, eight, nine, or ten RNA molecules (gRNA), DNA molecules, or expression vectors comprising a nucleic acid that encodes the gRNA, or any combination thereof.

In certain embodiments, the nuclease system can be CRISPR-Cas9.

In certain embodiments, the nuclease system inactivates or excises gene mutations.

In certain embodiments, the system further comprises a DNA double-stranded break (DSB) repair system.

In certain embodiments, the DSB repair system comprises a repair template in combination with or without a Non-Homologous End-Joining (NHEJ) or Homology Directed Repair (HDR) targeted to the one or more CRISPR-Cas9 cleavage site, said site corrects or edits a genomic mutation.

In certain embodiments, the DSB repair system is provided by the host cell machinery.

In certain embodiments, the genome targeted nuclease can be Cas9.

In certain embodiments, the disorder can be a corneal dystrophy or microsatellite expansion disease.

In certain embodiments, the ocular area can be the cornea.

In certain embodiments, the guide DNA comprises at least one, two, three, four, five, six, seven, eight, nine, or ten targeted genomic sequences.

In certain embodiments, the target genomic sequences are selected from any one of the nucleotide sequences set forth in SEQ ID NOs: 1-172 and 174-342, or any combination thereof.

In certain embodiments, the nuclease system can be administered topically to the surface of the eye.

In certain embodiments, the nuclease system can be administered on or outside the cornea, sclera, to the intraocular, subconjunctival, sub-tenon, or retrobulbar space, or in or around the eyelids.

In certain embodiments, the nuclease system can be administered by implantation, injection, or virally.

Another aspect of the invention relates to a method for treating a disorder affecting non-ocular tissue in a subject, the method comprising administering to the non-ocular tissue of the subject a therapeutically effective amount of a nuclease system comprising a genome targeted nuclease and a guide DNA comprising at least one targeted genomic sequence.

In certain embodiments, the nuclease can be provided as a protein, RNA, DNA, or an expression vector comprising a nucleic acid encoding the nuclease.

In certain embodiments, the guide DNA can be provided as an RNA molecule (gRNA), DNA molecule, or an expression vector comprising a nucleic acid that encodes the gRNA.

In certain embodiments, the nuclease system can be CRISPR-Cas9.

In certain embodiments, the nuclease system inactivates or excises gene mutations.

In certain embodiments, the method further comprises a DNA double-stranded break (DSB) repair system.

In certain embodiments, the DSB repair system comprises a repair template in combination with a Non-Homologous End-Joining (NHEJ) or Homology Directed Repair (HDR) targeted to the one or more CRISPR-Cas9 cleavage site, said site corrects or edits a genomic mutation.

In certain embodiments, the genome targeted nuclease can be Cas9.

In certain embodiments, the disorder can be microsatellite expansion disease.

In certain embodiments, the guide DNA comprises at least one, two, three, four, five, six, seven, eight, nine, or ten targeted genomic sequences.

In certain embodiments, the target genomic sequences are selected from any one of the nucleotide sequences set forth in SEQ ID NOs: 1-172 and 174-342, or any combination thereof.

In certain embodiments, the nuclease system is administered topically, intravascularly, intradermally, transdermally, parenterally, intravenously, intramuscularly, intranasally, subcutaneously, regionally, percutaneously, intratracheally, intraperitoneally, intraarterially, intravesically, intratumorally, inhalationly, perfusionly, lavagely, directly via injection, or orally via administration and formulation.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 contains four panels (A)-(D) describing two identified sites as targetable by Cas9 using the gRNA sequences that overlap with the respective mutations and their ability to disrupt dominant mutations in genes known to be causative in corneal dystrophies. Panel (A) depicts targeting of TGFBI exon 124 in HEK293 cells using the CRISPR-Cas9 system. The % gene modification by non-homologous end-joining (% indel) is indicated below. Panel (B) depicts an image trace of the gel indicating the peaks used for quantification. Panel (C) depicts targeting of TGFBI exon 555 in HEK293 cells using the CRISPR-Cas9 system. The % gene modification by non-homologous end-joining (% indel) is indicated below. Panel (D) depicts an image trace of the gel indicating the peaks used for quantification.

FIG. 2 contains three panels (A)-(C) describing identified sites as targetable by Cas9 using the gRNA sequences that correspond to target sequences within the intron between exon 2 and exon 3 of the TCF4 gene. Panel (A) depicts in HEK293 cells using the CRISPR/Cas9 system 6 gRNAs targeting intronic sequences downstream (Table 4) of the trinucleotide repeat expansion which causes Fuchs corneal dystrophy. Molecular weight ladder is shown in the far left and far right lanes. Control lane indicates no gRNA and no Cas9 transfection. Cas9 lane indicates transfection with Cas9 but no gRNA. Arrows indicate major cleavage product produced by non-homologous end-joining, and % gene modification by non-homologous end-joining is indicated below. Panel (B) depicts image traces of the gel indicating the peaks used for quantification. Panel (C) depicts expected digest sizes for each gRNA.

FIG. 3 contains three panels (A)-(C) describing identified sites as targetable by Cas9 using the gRNA sequences that correspond to target sequences within the intron between exon 2 and exon 3 of the TCF4 gene. Panel (A) depicts in HEK293 cells using the CRISPR/Cas9 system 6 gRNAs targeting intronic sequences upstream (Table 3) of the trinucleotide repeat expansion which causes Fuchs corneal dystrophy. Molecular weight ladder is shown in the far right lane. Control lane indicates no gRNA and no Cas9 transfection. Arrows indicate major cleavage products produced by non-homologous end-joining, and % gene modification by non-homologous end-joining is indicated below. Panel (B) depicts image traces of the gel indicating the peaks used for quantification. Panel (C) depicts expected digest sizes for each gRNA.

### DETAILED DESCRIPTION

Described herein are methods for treating eye disorders, such as corneal dystrophies and microsatellite expansion diseases. The methods use a nuclease system, such as Clustered Regularly Interspaced Short Palindromic Repeats (CRISPR)/CRISPR associated (Cas) 9 (CRISPR-Cas9), to cut, nick, and/or repair genomic DNA.

As used herein, the term “eye disease” may encompass disorders of the eye including, but not limited to corneal dystrophies and microsatellite expansion diseases.

As used herein, the term “corneal dystrophy” or “corneal dystrophies” describes a group of disorders that are generally inherited, bilateral, symmetric, slowly progressive, and not predominantly related to environmental or systemic factors (1,2). Corneal dystrophies, include (but may not be limited to) the following: Epithelial Basement Membrane Dystrophy (aka Map-Dot-Fingerprint Dystrophy, Cogan Microcystic Epithelial Dystrophy, Anterior Basement Membrane Dystrophy); Epithelial Recurrent Erosion Dystrophies (aka Franceschetti Corneal Dystrophy, Dystrophia Smolandiensis, Dystrophia Helsinglandica); Subepithelial Mucinous Corneal Dystrophy; Meesmann Corneal Dystrophy (aka Juvenile Hereditary Epithelial Dystrophy, Stocker Holt Dystrophy); Lisch Epithelial Corneal Dystrophy (aka Band-Shaped and Whorled Microcystic Dystrophy); Gelatinous Drop-like Corneal Dystrophy (aka Subepithelial Amyloidosis, Primary Familial Amyloidosis (of Grayson)); Reis-Bucklers Corneal Dystrophy (aka Corneal Dystrophy of Bowman layer, type I (CDB I), Geographic Corneal Dystrophy (of Weidle), Atypical Granular Corneal Dystrophy, Granular Corneal Dystrophy, Type 3, Anterior Limiting Membrane Dystrophy, Type 1, Superficial Granular Corneal Dystrophy); Thiel-Behnke Corneal Dystrophy (aka Corneal Dystrophy of Bowman layer, Type II (CDB2), Honeycomb-Shaped Corneal Dystrophy, Anterior Limiting Membrane Dystrophy, Type II, Curly Fibers Corneal Dystrophy, Waardenburg-Jonkers Corneal Dystrophy); Lattice Corneal Dystrophy, Type 1 (Classic) (aka Biber-Haab-Dimmer Dystrophy); Lattice Corneal Dystrophy, Type 2 (aka Familial Amyloidosis (Finnish Type or Gelsolin Type), Meretoja Syndrome); Lattice Corneal Dystrophy, Type III; Lattice Corneal Dystrophy, Type IIIA; Lattice Corneal Dystrophy, Type I/IIIA; Lattice Corneal Dystrophy, Type IV; Polymorphic (Corneal) Amyloidosis; Granular Corneal Dystrophy, Type 1 (aka Corneal Dystrophy Groenouw Type I); Granular Corneal Dystrophy, Type 2 (aka Avellino Dystrophy, Combined Granular-Lattice Dystrophy); Macular Corneal Dystrophy (aka Groenouw Corneal Dystrophy Type II, Fehr Speckled Dystrophy); Schnyder Corneal Dystrophy (aka Schnyder Crystalline Corneal Dystrophy (SCCD), Schnyder Crystalline Dystrophy Sine Crystals, Hereditary Crystalline Stromal Dystrophy of Schnyder, Crystalline Stromal Dystrophy, Central Stromal Crystalline Corneal Dystrophy, Corneal Crystalline Dystrophy of Schnyder, Schnyder Corneal Crystalline Dystrophy); Congenital Stromal Corneal Dystrophy (aka Congenital Hereditary Stromal Dystrophy); Fleck Corneal Dystrophy (aka François-Neetens Speckled (Mouchetée) Corneal Dystrophy); Posterior Amorphous Corneal Dystrophy (aka Posterior Amorphous Stromal Dystrophy); Central Cloudy Dystrophy of Francois; Pre-Descemet Corneal Dystrophy; Fuchs Endothelial Corneal Dystrophy (aka Endoepithelial Corneal Dystrophy); Posterior Polymorphous Corneal Dystrophy (aka Posterior Polymorphous Dystrophy, Schlichting Dystrophy); Congenital Hereditary Endothelial Dystrophy (aka Maumenee Corneal Dystrophy); X-linked Endothelial Corneal Dystrophy.

All of the above disorders are caused by known or putative genetic mutations. Corneal dystrophies yet to be described will be caused by known or putative genetic mutations. Thus, all genetic corneal dystrophies can be amenable to the nuclease system, like CRISPR-Cas9, for gene therapy involving correction or inactivation of the mutant allele.

As used herein, “microsatellite sequences”, also called short tandem repeats, are short DNA sequences (usually 2-5 nucleotides) which are repeated, typically in the range of 5-50 times. These sequences are present throughout the human genome and can become mutated and/or increased in the number of repeats. Some microsatellite sequences, if they expand beyond a certain length, can result in microsatellite expansion diseases. All known or yet to be described microsatellite expansion diseases will be caused by expansions in known or putative genes. Thus, all microsatellite expansion diseases can be amenable to CRISPR-Cas9 gene therapy involving correction or inactivation of the mutant allele.

Microsatellite expansion diseases as used herein may encompasses diseases that affect ocular and non-ocular tissues, including (but may not be limited to) the following disorders: Blepharophimosis, ptosis and epicanthus inversus syndactyly; Cleidocranial dysplasia; Congenital central hypoventilation syndrome, Haddad syndrome DM (Myotonic dystrophy); FRAXA (Fragile X syndrome); FRAXE (Fragile XE mental retardation); FRDA (Friedreich's ataxia); Fuchs' Endothelial Corneal Dystrophy; FXTAS (Fragile X-associated tremor/ataxia syndrome); Hand-foot-genital syndrome; HD (Huntington's disease); Holoprosencephaly; Mental retardation with growth hormone deficiency; Mental retardation, epilepsy, West syndrome, Partington syndrome; Oculopharyngeal muscular dystrophy; SBMA (Spinal and bulbar muscular atrophy); SCA1 (Spinocerebellar ataxia Type 1); SCA12 (Spinocerebellar ataxia Type 12); SCA17 (Spinocerebellar ataxia Type 17); SCA2 (Spinocerebellar ataxia Type 2); SCA3 (Spinocerebellar ataxia Type 3 or Machado-Joseph disease); SCA6 (Spinocerebellar ataxia Type 6); SCAT (Spinocerebellar ataxia Type 7); SCA8 (Spinocerebellar ataxia Type 8); Synpolydactyly.

As used herein, the term “eye”, “eye area” or “ocular area” of the subject encompasses the cornea, conjunctiva, sclera, fovea, macula, optic nerve, retina, lens, iris, pupil, to the intraocular, subconjunctival, sub-tenon, or retrobulbar space, or in or around the eyelids, and other anatomical features of the eye.

As used herein the Clustered Regularly Interspaced Short Palindromic Repeats (CRISPR)/CRISPR associated (Cas) 9 nuclease are an extremely versatile and accurate approach to cut and/or repair genomic DNA (6). CRISPR-Cas9-based gene editing can be used to inactivate or correct gene mutations causing corneal dystrophies and microsatellite expansion diseases, thereby providing a gene therapy approach for these groups of diseases. The naturally occurring CRISPR system from S. pyogenes has been modified to utilize a single guide RNA (gRNA) consisting of a 20 nucleotide (nt) target sequence and an additional structural RNA portion which binds the Cas9 double strand nuclease (6,7). The CRISPR-Cas9 system from S. pyogenes has the potential to cut at any 20 nt sequence adjacent to a 5′-NGG-3′ protospacer-adjacent motif (PAM), or alternate PAM sequences and bioinformatics provides tools to map target sites (8, 10). DNA cut by Cas9 is repaired by endogenous cellular mechanisms, including non-homologous end-joining (NHEJ), which produces insertion deletion mutations that can inactivate the original mutant allele. Thus, CRISPR-Cas9 can correct disease causing genetic mutations by cutting DNA in close enough proximity to a protein coding mutation to inactivate it through frameshifting. Alternatively, CRISPR-Cas9 can correct disease causing genetic mutations, either coding or non-coding, by cutting DNA on both sides of a mutation to excise it, or nicking on different strands flanking the mutation or repeat, if the distance is under 200 bp or so, or through the use of a repair template and homology directed repair (HDR) targeted to one or more CRISPR-Cas9 cleavage sites. Thus, specific mutant sequences can be gene edited and repaired.

CRISPR-Cas9 applied to corneal cells can correct the genetic defect causing corneal dystrophies and thus be used to treat these disorders. The CRISPR-Cas9 treatment could be administered topically to the surface of the eye, via implant, or via injection. The implant or injection could be administered to the cornea, sclera, to the intraocular, subconjunctival, sub-tenon, or retrobulbar space, or in or around the eyelids. CRISPR-Cas9 can also be applied outside the cornea or eye to treat other microsatellite expansion diseases in addition to Fuchs endothelial corneal dystrophy. CRISPR-Cas9 approaches to treat corneal dystrophies and microsatellite expansion diseases could employ single or multiple guide RNAs to inactivate or excise gene mutations, or using a repair template to correct gene mutations. In other embodiments, the CRISPR-Cas9 treatment may be applied to non-ocular tissue to correct the genetic defect causing microsatellite expansion diseases.

In certain embodiments, the routes of CRISPR-Cas9 treatment administration can vary with the location and nature of the cells or tissues to be contacted, and include, e.g., intravascular, intradermal, transdermal, parenteral, intravenous, intramuscular, intranasal, subcutaneous, regional, percutaneous, intratracheal, intraperitoneal, intraarterial, intravesical, intratumoral, inhalation, perfusion, lavage, direct injection, and oral administration and formulation, or any of the following routes of administration. The term “systemic administration” refers to administration in a manner that results in the introduction of the composition into the subject's circulatory system or otherwise permits its spread throughout the body. “Regional” administration refers to administration into a specific, and somewhat more limited, anatomical space, such as intraperitoneal, intrathecal, subdural, or to a specific organ. “Local administration” refers to administration of a composition or drug into a limited, or circumscribed, anatomic space, such as intratumoral injection into a tumor mass, subcutaneous injections, intradermal or intramuscular injections. Those of skill in the art will understand that local administration or regional administration may also result in entry of a composition into the circulatory system i.e., rendering it systemic to one degree or another. For example, the term “intravascular” is understood to refer to delivery into the vasculature of a patient, meaning into, within, or in a vessel or vessels of the patient, whether for systemic, regional, and/or local administration. In certain embodiments, the administration can be into a vessel considered to be a vein (intravenous), while in others administration can be into a vessel considered to be an artery. Veins include, but are not limited to, the internal jugular vein, a peripheral vein, a coronary vein, a hepatic vein, the portal vein, great saphenous vein, the pulmonary vein, superior vena cava, inferior vena cava, a gastric vein, a splenic vein, inferior mesenteric vein, superior mesenteric vein, cephalic vein, and/or femoral vein. Arteries include, but are not limited to, coronary artery, pulmonary artery, brachial artery, internal carotid artery, aortic arch, femoral artery, peripheral artery, and/or ciliary artery. It is contemplated that delivery may be through or to an arteriole or capillary.

The CRISPR-Cas system may be used facilitate targeted genome editing in eukaryotic cells, including mammalian cells, such as human cells. To facilitate genome editing, the cell to be modified is co-transfected with an expression vector encoding Cas9 or the Cas9 protein, DNA, or RNA itself, along with a guide-RNA molecule itself, or an expression vector comprising a nucleic acid molecule encoding the guide-RNA molecule. For example, in certain embodiments, the introduction of Cas9 can be done by transfecting in Cas9 as a protein, RNA, DNA, or expression vector comprising a nucleic acid that encodes Cas9. In certain embodiments, the guide DNA can itself be administered directly as an RNA molecule (gRNA), DNA molecule, or as expression vector comprising a nucleic acid that encodes the gRNA.

While many different CRISPR-Cas systems could be modified to facilitate targeted genome modification, the most commonly used CRISPR-Cas system in targeted genome modification is the CRISPR-Cas9 system from S. pyogenes. The CRISPR-Cas9 system requires only a single protein, Cas9, to catalyze double-stranded DNA breaks at sites targeted by a guide-RNA molecule.

Multiple guide RNA sequences can be encoded in a single CRISPR array to facilitate the simultaneous editing of multiple sites within a cell's genome. For example, a pair of guide RNAs can target proximally located sequences to facilitate the deletion of the intervening sequence. In some embodiments, Cas9 is encoded by a codon-optimized sequence. Plasmids encoding Cas9, including codon-optimized plasmids and plasmids encoding engineered Cas9 nickase are publicly available from Addgene (http://www.addgene.org/CRISPR/).

Additional information on the application of CRISPR-Cas systems to targeted genome engineering can be found in Jinek et al., Science 337:816-821 (2012); Cho et al., Nature Biotechnology 31:230-232 (2013); Cong et al., Science 339:819-823 (2013); Jinek et al., eLife 2:e00471 (2013); Mali et al., Science 339:823-826 (2013); Qi et al., Cell 152:1173-1183 (2013); Fu et al., Nature Biotechnology 31:822-826 (2013); Fu et al., Nature Biotechnology 31:822-826 (2013); Hsu et al., Nature Biotechnology 31:827-832 (2013); Mali et al., Nature Biotechnology 31:833-838 (2013); Pattanayak et al., Nature Biotechnology 31:839-843 (2013) and WO/2013/142578, each of which is hereby incorporated by reference in its entirety.

In some embodiments of the methods provided herein, the target nucleic acid sequence is modified using a CRISPR/Cas system. In some embodiments, the CRISPR/Cas system is a CRISPR-Cas9 system. In some embodiments, the subject is administered a nucleic acid encoding Cas9 and a nucleic acid encoding a guide-RNA that is specific to a target nucleic acid sequence in the eye.

In some embodiments, the guide-RNA comprises a target-specific guide sequence (e.g., a sequence that is complementary to a sequence of the target DNA sequence) and a guide-RNA scaffold sequence. In some embodiments, the target-specific guide sequence is a nucleic acid sequence selected from any one of SEQ ID NOs: 1-172 and 174-342, or any combination thereof. The target-specific guide sequence may comprise two, three, four, five, six, seven, eight, nine, ten, eleven, twelve, thirteen, fourteen, fifteen, sixteen, seventeen, eighteen, nineteen, or twenty nucleic acid sequences selected from the nucleotide sequences set forth in SEQ ID NOs: 1-172 and 174-342.

Having now fully described this invention, it will be appreciated by those skilled in the art that the same can be performed within a wide range of equivalent parameters, concentrations, and conditions without departing from the spirit and scope of the invention and without undue experimentation.

The present description is further illustrated by the following examples, which should not be construed as limiting in any way.

### Examples

CRISPR-Cas9 guide RNAs (gRNAs) targeting known mutations causing corneal dystrophies were identified (Table 1a-1c). Human genomic sequences corresponding to gRNA IDs in Table 1 are listed in Table 2.

Mutations in transforming growth factor beta-induced (TGFBI) gene are known to cause several forms of corneal dystrophies including Reis-Bücklers corneal dystrophy, Thiel-Behnke corneal dystrophy, Lattice corneal dystrophy, Granular corneal dystrophy, type 1, and Granular corneal dystrophy, type 2 (1). Missense mutations at two hotspots, R124 and R555, account for nearly 50% of the TGFBI-related corneal dystrophies (9).

In order to demonstrate the feasibility of CRISPR-based treatments for corneal dystrophies, two Cas9 targeting sites were identified that overlap with the genomic sequence encoding both R124 and R555 of TGFBI: 5′-TCAGCTGTACACGGACCGCACGG-3′ (SEQ ID NO: 145), and 5′-AGAGAACGGAGCAGACTCTTGGG-3′(SEQ ID NO: 171), located in exons 4 and 12, respectively. Specific amino acid substitutions at these residues result in clinically distinct corneal dystrophies: R124C—Lattice corneal dystrophy, type I; R124H—Granular corneal dystrophy, type 2; R555W—Granular corneal dystrophy, type 1; and R555Q—Reis-Bücklers corneal dystrophy.

The two target sites were cloned in pH1v1 (Addgene 60244) as described (8), and HEK293 cells were co-transfected with Cas9 and guide RNA (gRNA) constructs. Forty-eight or sixty hours post transfection, genomic DNA was harvested and the sequence surrounding the target cut sites were amplified according to the primers listed in the Appendix A (see below). The PCR products were then purified and quantified before performing the T7 Endo I assay.

Briefly, 200 ng of PCR product was denatured and then slowly re-annealed to allow for the formation of heteroduplexes, T7 Endonuclease I was added to the PCR products and incubated at 37° C. for 25/30 minutes to cleave heteroduplexes. The reaction was stopped by putting PCR products on ice, purified and finally run on a 6% TBE PAGE gel to resolve the products. The gel was stained with SYBR-Gold/Diamond Nucleic Acid dye from Promega, visualized, and quantified using ImageJ. Non-homologous end joining (NHEJ) frequencies were calculated using the binomial-derived equation:

$$\% gene modification = 1 - \sqrt{1 - \frac{( a + b )}{( a + b + c )}} \times 100 ;$$

where the values of “a” and “b” are equal to the integrated area of the cleaved fragments after background subtraction and “c” is equal to the integrated area of the un-cleaved PCR product after background subtraction.

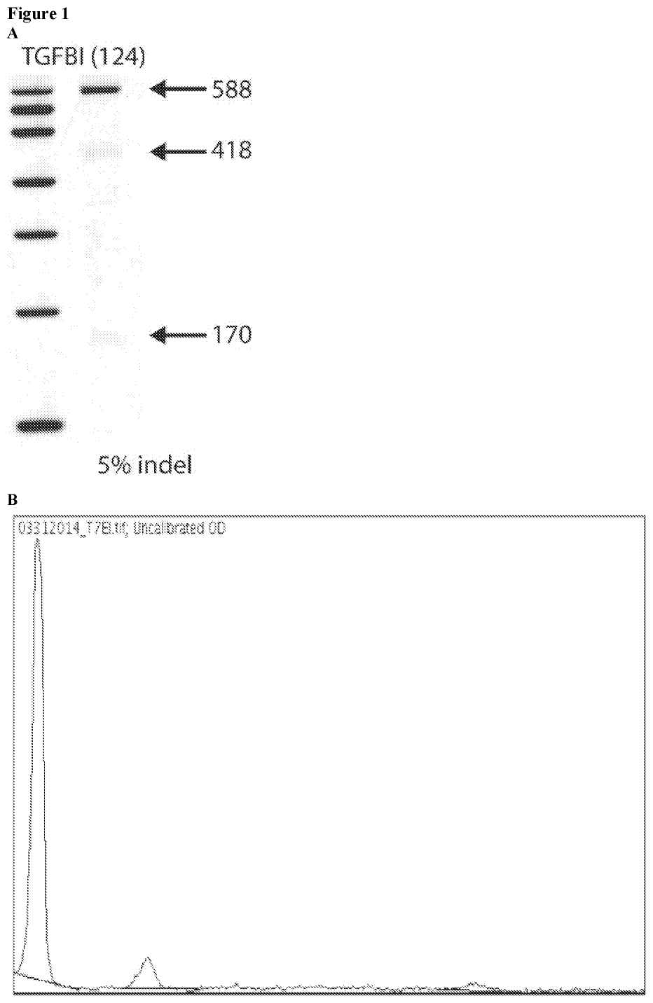

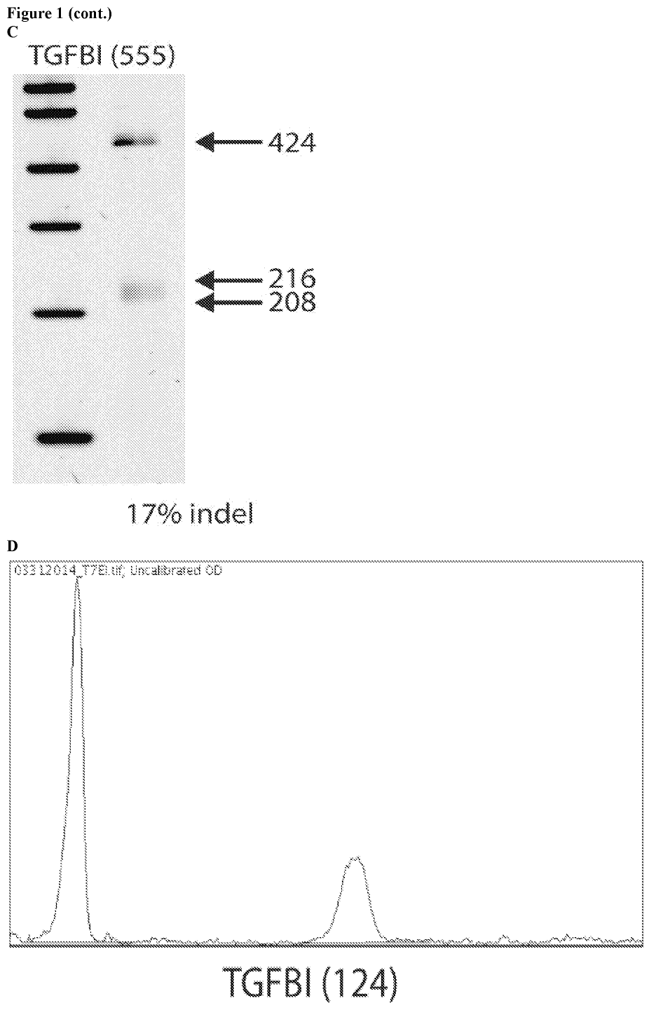

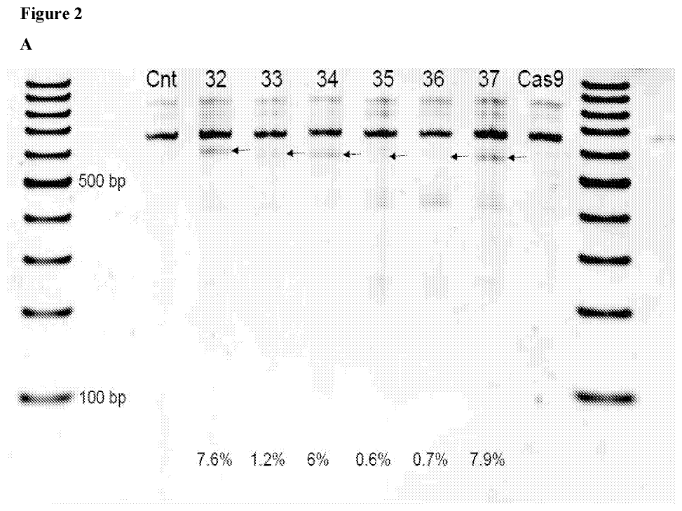

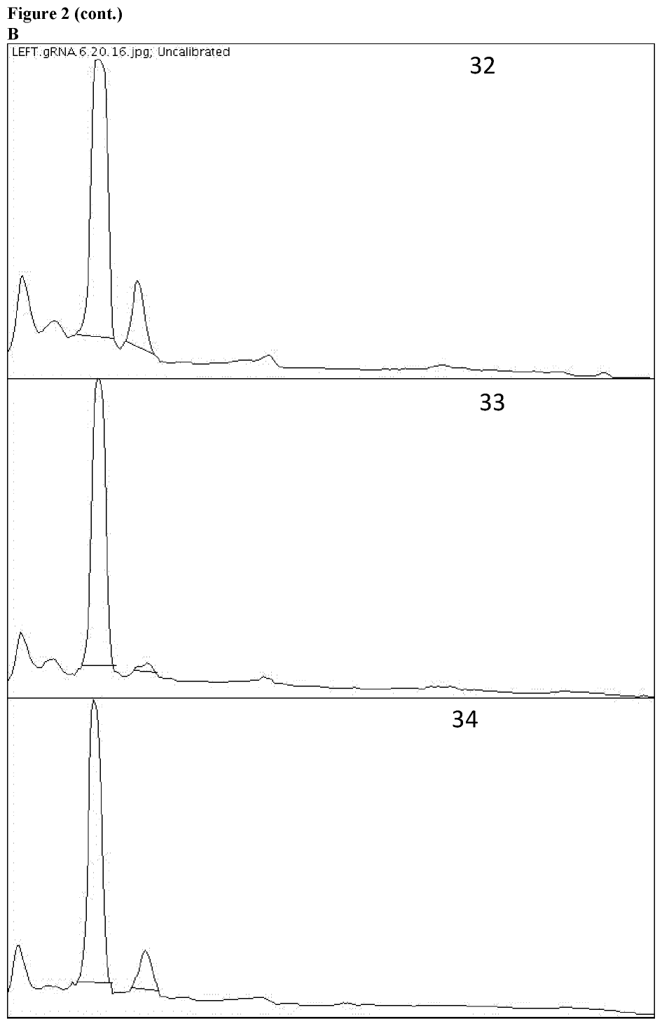

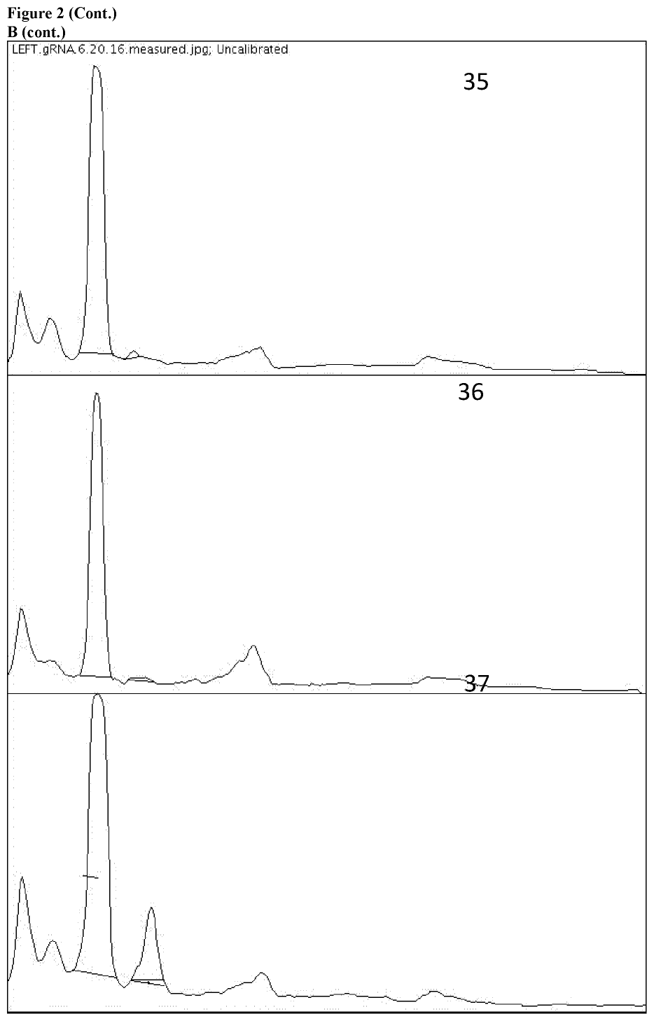

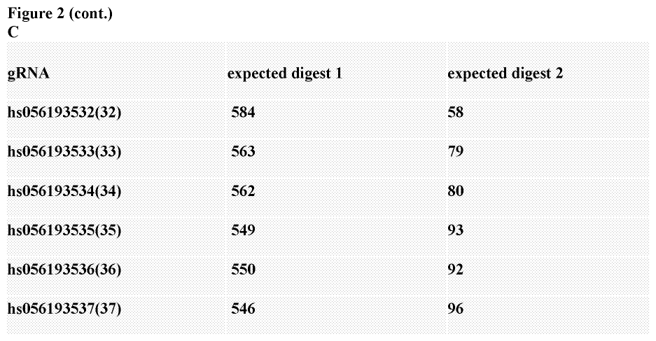

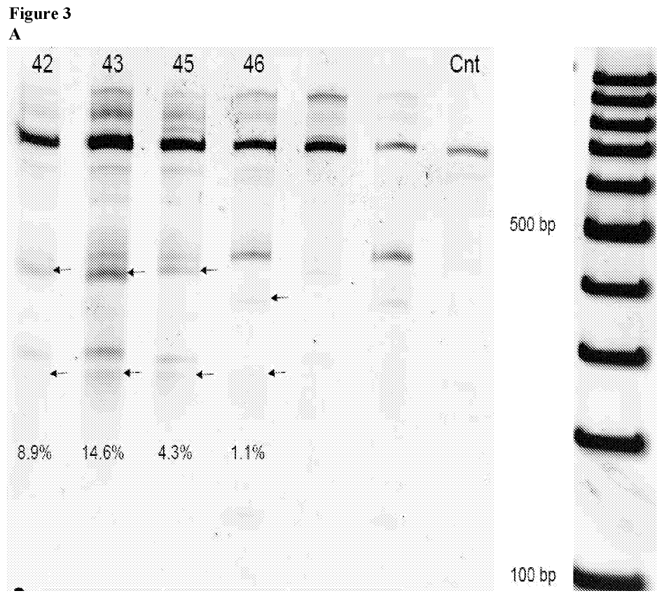

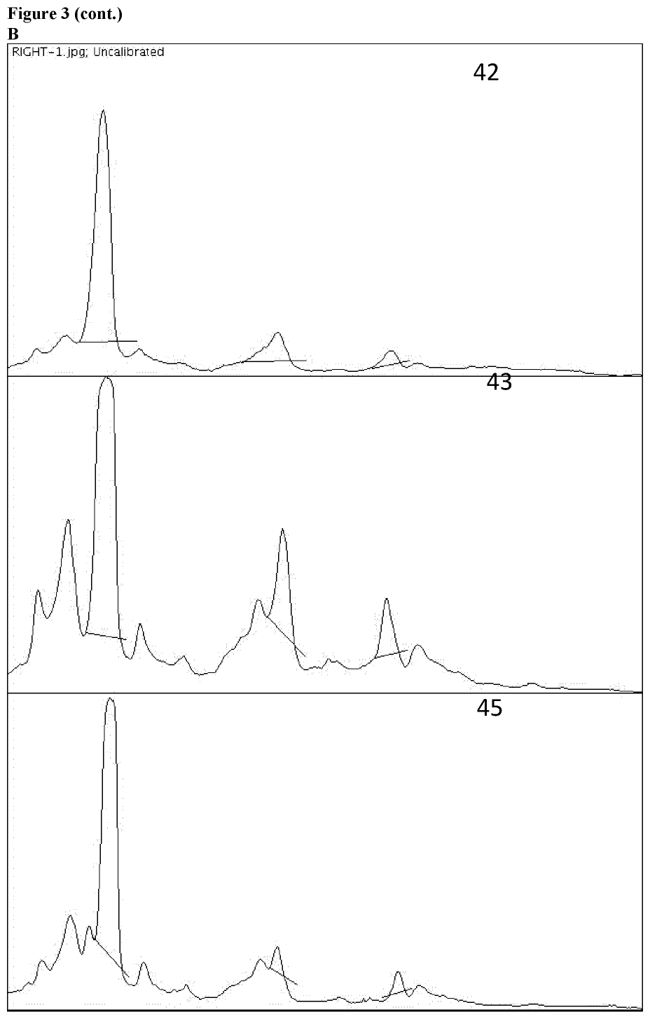

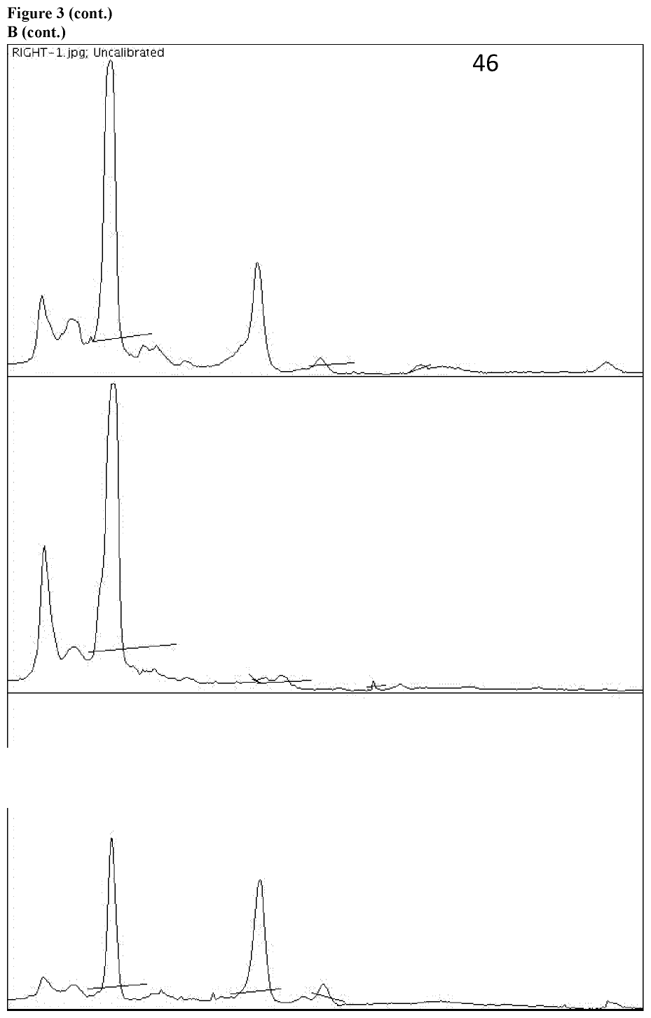

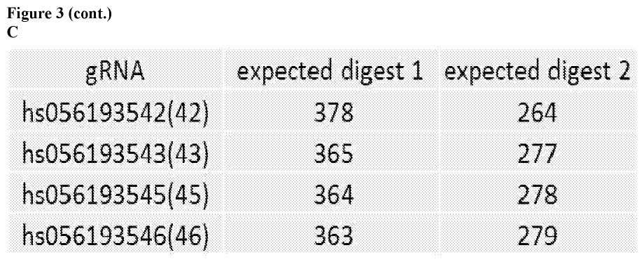

The results (FIG. 1) indicate that all identified sites were targetable by Cas9 using the gRNA sequences that either overlap with the respective mutations (TGFB1) or targets 5′ or 3′ of the repeat region (TCF4). These results demonstrate the ability to disrupt dominant mutations in genes known to be causative in corneal dystrophies.

CRISPR-Cas9 approaches to treat corneal dystrophies and microsatellite expansion diseases could employ single or multiple guide RNAs to inactivate or excise gene mutations, or using a repair template and homology directed repair to correct a gene mutation. In the case of the TCF4 microsatellite expansion causing FECD, one or more gRNAs targeting a region on one side of a microsatellite expansion or regions on both sides of a microsatellite expansion could be used. Table 3 shows IDs and corresponding human genomic sequences for gRNA target sequences upstream of the TCF4 microsatellite expansion causing FECD. Table 4 shows IDs and corresponding human genomic sequences for gRNA target sequences downstream of the same TCF4 microsatellite expansion. These gRNAs or others in the TCF4 gene could be used in any combination to correct the microsatellite expansion causing FECD. A similar approach using one or more gRNAs targeting a region on one side of a microsatellite expansion or regions on both sides of a microsatellite expansion could be used for other microsatellite expansion diseases, including but not limited to those listed in Table 5.

APPENDIX A
CRISPR Targets:
TGFBI (124)
hs101533615: TCAGCTGTACACGGACCGCACGG (SEQ ID NO: 145)
TGFBI (555)
hs101534962: AGAGAACGGAGCAGACTCTTGGG (SEQ ID NO: 171)
TCF4 (downstream of trinucleotide repeat)
hs056193532-AAGTGCAACAAGCAGAAAGGGGG (SEQ ID NO: 333)
hs056193533-GGCTGCAAAGCTGCCTGCCTAGG (SEQ ID NO: 334)
hs056193534-GCTGCAAAGCTGCCTGCCTAGGG (SEQ ID NO: 335)
hs056193535-CTGCCTAGGGCTACGTTTCCTGG (SEQ ID NO: 336)
hs056193536-CAGGAAACGTAGCCCTAGGCAGG (SEQ ID NO: 337)
hs056193537-TTGCCAGGAAACGTAGCCCTAGG (SEQ ID NO: 338)
TCF4 (upstream of trinucleotide repeat)
hs056193542-AAAGAGCCCCACTTGGAAGGCGG (SEQ ID NO: 195)
hs056193543-GCCCCACTTGGAAGGCGGTTTGG (SEQ ID NO: 196)
hs056193545-TCCAAACCGCCTTCCAAGTGGGG (SEQ ID NO: 198)
hs056193546-ATCCAAACCGCCTTCCAAGTGGG (SEQ ID NO: 199)
gRNA Primers:
TGFBI (124) humanF; CTTATAAGTTCTGTATGAGACCACTTTTTCCCTCAGCT
GTACACGGACCGCAG (SEQ ID NO: 173)
TGFBI (124) humanR; CCTTATTTTAACTTGCTATTTCTAGCTCTAAAACTGCG
GTCCGTGTACAGCTGAGG (SEQ ID NO: 343)
TGFBI (555) humanF; CTTATAAGTTCTGTATGAGACCACTTTTTCCCAGAGA
ACGGAGCAGACTCTTG (SEQ ID NO: 344)
TGFBI (555) humanR; CCTTATTTTAACTTGCTATTTCTAGCTCTAAAACAAG
AGTCTGCTCCGTTCTCTGG (SEQ ID NO: 345)
hs537_H1for; CTTATAAGTTCTGTATGAGACCACTTTTTCCCTTGCCAGGAA
ACGTAGCCCTG (SEQ ID NO: 352)
hs537_H1rev; CCTTATTTTAACTTGCTATTTCTAGCTCTAAAACAGGGCTAC
GTTTCCTGGCAAG (SEQ ID NO: 353)
hs536_H1for; CTTATAAGTTCTGTATGAGACCACTTTTTCCCCAGGAAACGT
AGCCCTAGGCG (SEQ ID NO: 354)
hs536_H1rev; CCTTATTTTAACTTGCTATTTCTAGCTCTAAAACGCCTAGGG
CTACGTTTCCTGG; (SEQ ID NO: 355)
hs535_H1for; CTTATAAGTTCTGTATGAGACCACTTTTTCCCCTGCCTAGGG
CTACGTTTCCG (SEQ ID NO: 356)
hs535_H1rev; CCTTATTTTAACTTGCTATTTCTAGCTCTAAAACGGAAACGT
AGCCCTAGGCAGG (SEQ ID NO: 357)
hs534_H1for; CTTATAAGTTCTGTATGAGACCACTTTTTCCCGCTGCAAAGC
TGCCTGCCTAG (SEQ ID NO: 358)
hs534_H1rev; CCTTATTTTAACTTGCTATTTCTAGCTCTAAAACTAGGCAGG
CAGCTTTGCAGCG (SEQ ID NO: 359)
hs533_H1for; CTTATAAGTTCTGTATGAGACCACTTTTTCCCGGCTGCAAAG
CTGCCTGCCTG (SEQ ID NO: 360)
hs533_H1rev; CCTTATTTTAACTTGCTATTTCTAGCTCTAAAACAGGCAGGC
AGCTTTGCAGCCG (SEQ ID NO: 361)
hs532_H1for; CTTATAAGTTCTGTATGAGACCACTTTTTCCCAAGTGCAACA
AGCAGAAAGGG (SEQ ID NO: 362)
hs532_H1rev; CCTTATTTTAACTTGCTATTTCTAGCTCTAAAACCCTTTCTGC
TTGTTGCACTTG (SEQ ID NO: 363)
hs542_H1for; CTTATAAGTTCTGTATGAGACCACTTTTTCCCAAAGAGCCCC
ACTTGGAAGGG (SEQ ID NO: 364)
hs542_H1rev; CCTTATTTTAACTTGCTATTTCTAGCTCTAAAACCCTTCCAA
GTGGGGCTCTTTG (SEQ ID NO: 365)
hs543_H1for; CTTATAAGTTCTGTATGAGACCACTTTTTCCCGCCCCACTTG
GAAGGCGGTTG (SEQ ID NO: 366)
hs543_H1rev; CCTTATTTTAACTTGCTATTTCTAGCTCTAAAACAACCGCCT
TCCAAGTGGGGCG (SEQ ID NO: 367)
hs545_H1for; CTTATAAGTTCTGTATGAGACCACTTTTTCCCTCCAAACCGC
CTTCCAAGTGG (SEQ ID NO: 368)
hs545_H1rev; CCTTATTTTAACTTGCTATTTCTAGCTCTAAAACCACTTGGA
AGGCGGTTTGGAG (SEQ ID NO: 369)
hs546_H1for; CTTATAAGTTCTGTATGAGACCACTTTTTCCCATCCAAACCG
CCTTCCAAGTG (SEQ ID NO: 370)
hs546_H1rev; CCTTATTTTAACTTGCTATTTCTAGCTCTAAAACACTTGGAA
GGCGGTTTGGATG (SEQ ID NO: 371)
T7Endo I Genomic Amplification Primers:
TGFBI124.1F; CCACCTGTAGATGTACCGTGCTCTC (SEQ ID NO: 346)
TGFBI124.1R; AGGGGCTGCAGACTCTGTGTTTAAG (SEQ ID NO: 347)
TGFBI555.1F; AAGGAAAATACCTCTCAGCGTGGTG (SEQ ID NO: 348)
TGFBI555.1R; AGGCCTAGGGGTAGTAAAGGCTTCC (SEQ ID NO: 349)
TCF4.3F: TGCTTTGGATTGGTAGGACCTGTTC (SEQ ID NO: 372)
TCF4.3R: GGATAATGCACACCTTCCCTGAGTC (SEQ ID NO: 373)
TGFBI exon 4 amplicon:
CCACCTGTAGATGTACCGTGCTCTCTGTCAGAGAAGGGAGGGTGTGGTTGGGCT
GGACCCCCAGAGGCCATCCCTCCTTCTGTCTTCTGCTCCTGCAGCCCTACCACTC
TCAAACCTTTACGAGACCCTGGGAGTCGTTGGATCCACCACCACTCAGCTGTAC
ACGGACCGCACGGAGAAGCTGAGGCCTGAGATGGAGGGGCCCGGCAGCTTCAC
CATCTTCGCCCCTAGCAACGAGGCCTGGGCCTCCTTGCCAGCTGTGAGATGACC
TCCGTCTGCCCGGGGGACTCTTATGGGGAACTGCCTTACTTCCCCGAGGGGTGG
GCATGATGAATGGGAGTCTGCAGTCATTTCCTACTGTTTCAGGAAGCTTTCTCCT
TAACCCCTTAGAAAAGGCTGTGGAACTTGAGCTAAAATATGTCTTACCAGGTTG
CGTCTAATGCCCCCCGTTCCCTACTGGGCAGAAAGACTTGGGTGCTTCCTGAGG
AGGGATCCTTGGCAGAAGAGAGGCCTGGGCTCACGAGGGCTGAGAACATGTTT
CCCAGAGTTGCAAGGACCCATCTCTTAAACACAGAGTCTGCAGCCCCT (SEQ ID
NO: 350)
TGFBI exon 12 amplicon:
AAGGAAAATACCTCTCAGCGTGGTGAGGTATTTAAGGAAAATACCTGTTGACA
GGTGACATTTTCTGTGTGTGTATCTACAGCATGCTGGTAGCTGCCATCCAGTCTG
CAGGACTGACGGAGACCCTCAACCGGGAAGGAGTCTACACAGTCTTTGCTCCC
ACAAATGAAGCCTTCCGAGCCCTGCCACCAAGAGAACGGAGCAGACTCTTGGG
TAAAGACCAACTTAAGTACACGTCTCCATTTTTCTAAAGTAGTGATCCCTCAGG
GCCCCAGCAGCAAACAGTTGGCACATCAAGGATTGACTTGAAGGGATTTTATG
ACAAGACTATTAGTGAAAGAGTGGGCGGGACTAAAGGAACTAGCAAAGGATG
AGGCCAACCAGGGACTAGCAACCCTGGGAAGCCTTTACTACCCCTAGGCCT
(SEQ ID NO: 351)
TCF4 gene amplicon:
TGCTTTGGATTGGTAGGACCTGTTCCTTACATCTTACCTCCTAGTTACATCTTTT
CCTAGGATTCTTAAAACTAGTATGGATATGCTGAGCATACATTCTTTAGAACCT
TTTGGACTGTTTTGGTAAATTTCGTAGTCGTAGGATCAGCACAAAGCGGAACTT
GACACACTTGTGGAGTTTTACGGCTGTACTTGGTCCTTCTCCATCCCTTTGCTTC
CTTTTCCTAAACCAAGTCCCAGACATGTCAGGAGAATGAATTCATTTTTAATGC
CAGATGAGTTTGGTGTAAGATGCATTTGTAAAGCAAAATAAAAAGAATCCACA
AAACACACAAATAAAATCCAAACCGCCTTCCAAGTGGGGCTCTTTCATGCTGCT
GCTGCTGCTGCTGCTGCTGCTGCTGCTGCTGCTGCTGCTGCTGCTGCTGCTGCTG
CTGCTGCTGCTGCTCCTCCTCCTCCTCCTCCTTCTCCTCCTCCTCCTCCTCTTCTA
GACCTTCTTTTGGAGAAATGGCTTTCGGAAGTTTTGCCAGGAAACGTAGCCCTA
GGCAGGCAGCTTTGCAGCCCCCTTTCTGCTTGTTGCACTTTCTCCATTCGTTCCT
TTGCTTTTTGCAGGCTCTGACTCAGGGAAGGTGTGCATTATCC (SEQ ID NO: 374)

TABLE 1a
Known gene mutations affecting the cornea and overlapping gRNA target
sequences by ID and gene(s).

| Gene ID: Mutation | Gene(s) |
|---|---|
| NM_005202.3(COL8A2): c.1363_1364delCAinsGT | COL8A2 |
| NM_005202.3(COL8A2): c.1363C > A (p.Gln455Lys) | COL8A2 |
| NM_005202.3(COL8A2): c.1349T > G (p.Leu450Trp) | COL8A2 |
| NM_001920.3(DCN): c.962delA (p.Lys321Argfs) | DCN |
| NM_001920.3(DCN): c.947delG (p.Gly316Aspfs) | DCN |
| NM_001920.3(DCN): c.967delT (p.Ser323Leufs) | DCN |
| NM_001920.3(DCN): c.941delC (p.Pro314Hisfs) | DCN |
| NM_000223.3(KRT12): c.55C > T (p.Arg19Trp) | KRT12 |
| NM_000223.3(KRT12): c.43C > T (p.Pro15Ser) | KRT12 |
| NM_000223.3(KRT12): c.427G > T (p.Val143Leu) | KRT12 |
| NM_000223.3(KRT12): c.409G > C (p.Ala137Pro) | KRT12 |
| NM_000223.3(KRT12): c.405A > C (p.Arg135Ser) | KRT12 |
| NM_000223.3(KRT12): c.399T > G (p.Asn133Lys) | KRT12 |
| NM_000223.3(KRT12): c.389A > C (p.Gln130Pro) | KRT12 |
| NM_000223.3(KRT12): c.385A > G (p.Met129Val) | KRT12 |
| NM_000223.3(KRT12): c.1298T > G (p.Leu433Arg) | KRT12 |
| NM_000223.3(KRT12): c.1289G > C (p.Arg430Pro) | KRT12 |
| NM_000223.3(KRT12): c.1286A > G (p.Tyr429Cys) | KRT12 |
| NM_000223.3(KRT12): c.1277T > G (p.Ile426Ser) | KRT12 |
| NM_000223.3(KRT12): c.1276A > G (p.Ile426Val) | KRT12 |
| NM_000223.3(KRT12): c.*360A > C | KRT12 |
| NM_000223.3(KRT12): c.386T > C (p.Met129Thr) | KRT12 |
| NM_000223.3(KRT12): c.419T > G (p.Leu140Arg) | KRT12 |
| NM_000223.3(KRT12): c.1285T > G (p.Tyr429Asp) | KRT12 |
| NM_000223.3(KRT12): c.404G > T (p.Arg135Ile) | KRT12 |
| NM_000223.3(KRT12): c.403A > G (p.Arg135Gly) | KRT12 |
| NM_000223.3(KRT12): c.427G > C (p.Val143Leu) | KRT12 |
| NM_057088.2(KRT3): c.109G > A (p.Gly37Arg) | KRT3 |
| NM_057088.2(KRT3): c.1347C > A (p.Ala449=) | KRT3 |
| NM_057088.2(KRT3): c.1508G > C (p.Arg503Pro) | KRT3 |
| NM_057088.2(KRT3): c.1493A > T (p.Glu498Val) | KRT3 |
| NM_057088.2(KRT3): c.1525G > A (p.Glu509Lys) | KRT3 |
| NM_015040.3(PIKFYVE): c.1370C > T (p.Ser457Phe) | PIKFYVE |
| NM_015040.3(PIKFYVE): c.5018T > A (p.Phe1673Tyr) | PIKFYVE |
| NM_015040.3(PIKFYVE): c.4167_4170delAGTA (p.Glu1389Aspfs*16) | PIKFYVE |
| NM_015040.3(PIKFYVE): c.2962C > T (p.Gln988Ter) | PIKFYVE |
| NM_015040.3(PIKFYVE): c.3308A > G (p.Lys1103Arg) | PIKFYVE |
| NM_000351.4(STS): c.1331A > G (p.His444Arg) | STS |
| NM_000351.4(STS): c.1115G > C (p.Trp372Ser) | STS |
| NM_000358.2(TGFBI): c.593C > T (p.Ser198Phe) | TGFBI |
| NM_000358.2(TGFBI): c.1998G > C (p.Arg666Ser) | TGFBI |
| NM_000358.2(TGFBI): c.1526T > G (p.Leu509Arg) | TGFBI |
| NM_000358.2(TGFBI): c.1619T > C (p.Phe540Ser) | TGFBI |
| NM_000358.2(TGFBI): c.370C > A (p.Arg124Ser) | TGFBI |
| TGFBI, 3-BP DEL | TGFBI |
| NM_000358.2(TGFBI): c.1868G > A (p.Gly623Asp) | TGFBI |
| NM_000358.2(TGFBI): c.371G > A (p.Arg124His) | TGFBI |
| NM_000358.2(TGFBI): c.370C > T (p.Arg124Cys) | TGFBI |
| NM_000358.2(TGFBI): c.1663C > T (p.Arg555Trp) | TGFBI |
| NM_000358.2(TGFBI): c.[1637C > A; 1652C > A] | TGFBI |
| NM_000358.2(TGFBI): c.371G > T (p.Arg124Leu) | TGFBI |
| NM_000358.2(TGFBI): c.1664G > A (p.Arg555Gln) | TGFBI |
| NM_000358.2(TGFBI): c.1501C > A (p.Pro501Thr) | TGFBI |
| NM_013319.2(UBIAD1): c.530G > A (p.Gly177Glu) | UBIAD1 |
| NM_013319.2(UBIAD1): c.708C > G (p.Asp236Glu) | UBIAD1 |
| NM_013319.2(UBIAD1): c.335A > G (p.Asp112Gly) | UBIAD1 |
| NM_013319.2(UBIAD1): c.355A > G (p.Arg119Gly) | UBIAD1 |
| NM_013319.2(UBIAD1): c.556G > A (p.Gly186Arg) | UBIAD1 |
| NM_013319.2(UBIAD1): c.511T > C (p.Ser171Pro) | UBIAD1 |
| NM_013319.2(UBIAD1): c.695A > G (p.Asn232Ser) | UBIAD1 |
| NM_013319.2(UBIAD1): c.524C > T (p.Thr175Ile) | UBIAD1 |
| NM_013319.2(UBIAD1): c.529G > C (p.Gly177Arg) | UBIAD1 |
| NM_013319.2(UBIAD1): c.305A > G (p.Asn102Ser) | UBIAD1 |
| NM_030751.5(ZEB1): c.2519A > C (p.Gln840Pro) | ZEB1 |
| NM_030751.5(ZEB1): c.233A > C (p.Asn78Thr) | ZEB1 |

TABLE 1b
Known gene mutations affecting the cornea and overlapping gRNA target
sequences by ID and condition(s).

| Gene ID: Mutation | Condition(s) |
|---|---|
| NM_005202.3(COL8A2): c.1363_1364delCAinsGT | Corneal dystrophy Fuchs |
| | endothelial 1 |
| NM_005202.3(COL8A2): c.1363C > A (p.Gln455Lys) | Corneal dystrophy Fuchs |
| | endothelial 1\|Corneal dystrophy, |
| | posterior polymorphous, 2 |
| NM_005202.3(COL8A2): c.1349T > G (p.Leu450Trp) | Corneal dystrophy Fuchs |
| | endothelial 1\|Corneal dystrophy, |
| | posterior polymorphous, 2 |
| NM_001920.3(DCN): c.962delA (p.Lys321Argfs) | Congenital Stromal Corneal |
| | Dystrophy |
| NM_001920.3(DCN): c.947delG (p.Gly316Aspfs) | Congenital Stromal Corneal |
| | Dystrophy |
| NM_001920.3(DCN): c.967delT (p.Ser323Leufs) | Congenital Stromal Corneal |
| | Dystrophy |
| NM_001920.3(DCN): c.941delC (p.Pro314Hisfs) | Congenital Stromal Corneal |
| | Dystrophy |
| NM_000223.3(KRT12): c.55C > T (p.Arg19Trp) | not provided |
| NM_000223.3(KRT12): c.43C > T (p.Pro15Ser) | not provided |
| NM_000223.3(KRT12): c.427G > T (p.Val143Leu) | not provided |
| NM_000223.3(KRT12): c.409G > C (p.Ala137Pro) | not provided |
| NM_000223.3(KRT12): c.405A > C (p.Arg135Ser) | not provided |
| NM_000223.3(KRT12): c.399T > G (p.Asn133Lys) | not provided |
| NM_000223.3(KRT12): c.389A > C (p.Gln130Pro) | not provided |
| NM_000223.3(KRT12): c.385A > G (p.Met129Val) | not provided |
| NM_000223.3(KRT12): c.1298T > G (p.Leu433Arg) | not provided |
| NM_000223.3(KRT12): c.1289G > C (p.Arg430Pro) | not provided |
| NM_000223.3(KRT12): c.1286A > G (p.Tyr429Cys) | not provided |
| NM_000223.3(KRT12): c.1277T > G (p.Ile426Ser) | not provided |
| NM_000223.3(KRT12): c.1276A > G (p.Ile426Val) | not provided |
| NM_000223.3(KRT12): c.*360A > C | not provided |
| NM_000223.3(KRT12): c.386T > C (p.Met129Thr) | Meesman's corneal dystrophy\|not |
| | provided |
| NM_000223.3(KRT12): c.419T > G (p.Leu140Arg) | Meesman's corneal dystrophy\|not |
| | provided |
| NM_000223.3(KRT12): c.1285T > G (p.Tyr429Asp) | Meesman's corneal dystrophy\|not |
| | provided |
| NM_000223.3(KRT12): c.404G > T (p.Arg135Ile) | Meesman's corneal dystrophy\|not |
| | provided |
| NM_000223.3(KRT12): c.403A > G (p.Arg135Gly) | Meesman's corneal dystrophy\|not |
| | provided |
| NM_000223.3(KRT12): c.427G > C (p.Val143Leu) | Meesman's corneal dystrophy\|not |
| | provided |
| NM_057088.2(KRT3): c.109G > A (p.Gly37Arg) | Malignant melanoma |
| NM_057088.2(KRT3): c.1347C > A (p.Ala449=) | Malignant melanoma |
| NM_057088.2(KRT3): c.1508G > C (p.Arg503Pro) | Meesman's corneal dystrophy\|not |
| | provided |
| NM_057088.2(KRT3): c.1493A > T (p.Glu498Val) | Meesman's corneal dystrophy\|not |
| | provided |
| NM_057088.2(KRT3): c.1525G > A (p.Glu509Lys) | Meesman's corneal dystrophy\|not |
| | provided |
| NM_015040.3(PIKFYVE): c.1370C > T (p.Ser457Phe) | Malignant melanoma |
| NM_015040.3(PIKFYVE): c.5018T > A | Malignant melanoma |
| (p.Phe1673Tyr) | |
| NM_015040.3(PIKFYVE): c.4167_4170delAGTA | Fleck corneal dystrophy |
| (p.Glu1389Aspfs*16) | |
| NM_015040.3(PIKFYVE): c.2962C > T (p.Gln988Ter) | Fleck corneal dystrophy |
| NM_015040.3(PIKFYVE): c.3308A > G | Fleck corneal dystrophy |
| (p.Lys1103Arg) | |
| NM_000351.4(STS): c.1331A > G (p.His444Arg) | X-linked ichthyosis with steryl- |
| | sulfatase deficiency |
| NM_000351.4(STS): c.1115G > C (p.Trp372Ser) | X-linked ichthyosis with steryl- |
| | sulfatase deficiency |
| NM_000358.2(TGFBI): c.593C > T (p.Ser198Phe) | Malignant melanoma |
| NM_000358.2(TGFBI): c.1998G > C (p.Arg666Ser) | Corneal epithelial dystrophy |
| NM_000358.2(TGFBI): c.1526T > G (p.Leu509Arg) | Corneal epithelial dystrophy |
| NM_000358.2(TGFBI): c.1619T > C (p.Phe540Ser) | Lattice corneal dystrophy type 3A |
| NM_000358.2(TGFBI): c.370C > A (p.Arg124Ser) | Groenouw corneal dystrophy type I |
| TGFBI, 3-BP DEL | Reis-Bucklers' corneal dystrophy |
| NM_000358.2(TGFBI): c.1868G > A (p.Gly623Asp) | Reis-Bucklers' corneal dystrophy |
| NM_000358.2(TGFBI): c.371G > A (p.Arg124His) | Avellino corneal dystrophy |
| NM_000358.2(TGFBI): c.370C > T (p.Arg124Cys) | Lattice corneal dystrophy Type I |
| NM_000358.2(TGFBI): c.1663C > T (p.Arg555Trp) | Groenouw corneal dystrophy type I |
| NM_000358.2(TGFBI): c.[1637C > A; 1652C > A] | Lattice corneal dystrophy Type I |
| NM_000358.2(TGFBI): c.371G > T (p.Arg124Leu) | Reis-Bucklers' corneal dystrophy |
| NM_000358.2(TGFBI): c.1664G > A (p.Arg555Gln) | Thiel-Behnke corneal dystrophy |
| NM_000358.2(TGFBI): c.1501C > A (p.Pro501Thr) | Lattice corneal dystrophy type 3A |
| NM_013319.2(UBIAD1): c.530G > A (p.Gly177Glu) | Schnyder crystalline corneal |
| | dystrophy |
| NM_013319.2(UBIAD1): c.708C > G (p.Asp236Glu) | Schnyder crystalline corneal |
| | dystrophy |
| NM_013319.2(UBIAD1): c.335A > G (p.Asp112Gly) | Schnyder crystalline corneal |
| | dystrophy |
| NM_013319.2(UBIAD1): c.355A > G (p.Arg119Gly) | Schnyder crystalline corneal |
| | dystrophy |
| NM_013319.2(UBIAD1): c.556G > A (p.Gly186Arg) | Schnyder crystalline corneal |
| | dystrophy |
| NM_013319.2(UBIAD1): c.511T > C (p.Ser171Pro) | Schnyder crystalline corneal |
| | dystrophy |
| NM_013319.2(UBIAD1): c.695A > G (p.Asn232Ser) | Schnyder crystalline corneal |
| | dystrophy |
| NM_013319.2(UBIAD1): c.524C > T (p.Thr175Ile) | Schnyder crystalline corneal |
| | dystrophy |
| NM_013319.2(UBIAD1): c.529G > C (p.Gly177Arg) | Schnyder crystalline corneal |
| | dystrophy |
| NM_013319.2(UBIAD1): c.305A > G (p.Asn102Ser) | Schnyder crystalline corneal |
| | dystrophy |
| NM_030751.5(ZEB1): c.2519A > C (p.Gln840Pro) | Corneal dystrophy, fuchs |
| | endothelial, 6 |
| NM_030751.5(ZEB1): c.233A > C (p.Asn78Thr) | Corneal dystrophy, fuchs |
| | endothelial, 6 |

TABLE 1c
Known gene mutations affecting the cornea and overlapping gRNA target
sequences by ID and gRNA(s).

| Gene ID: Mutation | gRNAs |
|---|---|
| NM_005202.3(COL8A2): c.1363_1364delCAinsGT | |
| NM_005202.3(COL8A2): c.1363 C > A (p.Gln455Lys) | hs002677511, hs002677512, |
| | hs002677505, hs002677513, |
| | hs002677506, hs002677516, |
| | hs002677517, hs002677510, |
| | hs002677518 |
| NM_005202.3(COL8A2): c.1349T > G (p.Leu450Trp) | hs002677507, hs002677508, |
| | hs002677509, hs002677511, |
| | hs002677512, hs002677505, |
| | hs002677513, hs002677506 |
| NM_001920.3(DCN): c.962delA (p.Lys321Argfs) | hs028999998, hs029000002, |
| | hs029000003, hs028999999, |
| | hs029000004, hs029000000 |
| NM_001920.3(DCN): c.947delG (p.Gly316Aspfs) | hs028999999, hs029000004, |
| | hs029000000, hs029000001, |
| | hs029000005 |
| NM_001920.3(DCN): c.967delT (p.Ser323Leufs) | hs028999998, hs029000002, |
| | hs029000003 |
| NM_001920.3(DCN): c.941delC (p.Pro314Hisfs) | hs029000000, hs029000001, |
| | hs029000005 |
| NM_000223.3(KRT12): c.55C > T (p.Arg19Trp) | hs051021143, hs051021144, |
| | hs051021148, hs051021149, |
| | hs051021150, hs051021151, |
| | hs051021154, hs051021155 |
| NM_000223.3(KRT12): c.43C > T (p.Pro15Ser) | hs051021148, hs051021149, |
| | hs051021150, hs051021151, |
| | hs051021154, hs051021155, |
| | hs051021157 |
| NM_000223.3(KRT12): c.427G > T (p.Val143Leu) | hs051021071, hs051021069, |
| | hs051021070 |
| NM_000223.3(KRT12): c.409G > C (p.Ala137Pro) | hs051021072, hs051021073 |
| NM_000223.3(KRT12): c.405A > C (p.Arg135Ser) | hs051021073 |
| NM_000223.3(KRT12): c.399T > G (p.Asn133Lys) | |
| NM_000223.3(KRT12): c.389A > C (p.Gln130Pro) | |
| NM_000223.3(KRT12): c.385A > G (p.Met129Val) | |
| NM_000223.3(KRT12): c.1298T > G (p.Leu433Arg) | hs051020754, hs051020755, |
| | hs051020748, hs051020750, |
| | hs051020752, hs051020756, |
| | hs051020758, hs051020753, |
| | hs051020759, hs051020760, |
| | hs051020763 |
| NM_000223.3(KRT12): c.1289G > C (p.Arg430Pro) | hs051020752, hs051020756, |
| | hs051020758, hs051020753, |
| | hs051020759, hs051020760, |
| | hs051020763 |
| NM_000223.3(KRT12): c.1286A > G (p.Tyr429Cys) | hs051020758, hs051020753, |
| | hs051020759, hs051020760, |
| | hs051020763 |
| NM_000223.3(KRT12): c.1277T > G (p.Ile426Ser) | hs051020757, hs051020761, |
| | hs051020762 |
| NM_000223.3(KRT12): c.1276A > G (p.Ile426Val) | hs051020757, hs051020761, |
| | hs051020762 |
| NM_000223.3(KRT12): c.*360A > C | |
| NM_000223.3(KRT12): c.386T > C (p.Met129Thr) | |
| NM_000223.3(KRT12): c.419T > G (p.Leu140Arg) | hs051021069, hs051021070, |
| | hs051021072, hs051021073 |
| NM_000223.3 (KRT12): c.1285T > G (p.Tyr429Asp) | hs051020758, hs051020753, |
| | hs051020759, hs051020760, |
| | hs051020763 |
| NM_000223.3(KRT12): c.404G > T (p.Arg135Ile) | hs051021073 |
| NM_000223.3(KRT12): c.403A > G (p.Arg135Gly) | hs051021073 |
| NM_000223.3(KRT12): c.427G > C (p.Val143Leu) | hs051021071, hs051021069, |
| | hs051021070 |
| NM_057088.2(KRT3): c.109G > A (p.Gly37Arg) | hs027503914, hs027503915, |
| | hs027503916, hs027503917, |
| | hs027503911, hs027503912, |
| | hs027503918, hs027503919, |
| | hs027503921 |
| NM_057088.2(KRT3): c.1347C > A (p.Ala449=) | hs027503292, hs027503297, |
| | hs027503293, hs027503299, |
| | hs027503294 |
| NM_057088.2(KRT3): c.1508G > C (p.Arg503Pro) | |
| NM_057088.2(KRT3): c.1493A > T (p.Glu498Val) | |
| NM_057088.2(KRT3): c.1525G > A (p.Glu509Lys) | |
| NM_015040.3(PIKFYVE): c.1370C > T (p.Ser457Phe) | hs070544033, hs070544034, |
| | hs070544030, hs070544031 |
| NM_015040.3(PIKFYVE): c.5018T > A (p.Phe1673Tyr) | hs070545604, hs070545605, |
| | hs070545601, hs070545606 |
| NM_015040.3(PIKFYVE): c.4167_4170delAGTA | hs070544848, hs070544849, |
| (p.Glu1389Aspfs*16) | hs070544850, hs070544847, |
| | hs070544851 |
| NM_015040.3(PIKFYVE): c.2962C > T (p.Gln988Ter) | hs070544887, hs070544888 |
| NM_015040.3(PIKFYVE): c.3308A > G | hs131603026, hs131603028 |
| (p.Lys1103Arg) | |
| NM_000351.4(STS): c.1331A > G (p.His444Arg) | hs131602458, hs131602459, |
| | hs131602461, hs131602462, |
| | hs131602463, hs131602464 |
| NM_000351.4(STS): c.1115G > C (p.Trp372Ser) | |
| NM_000358.2(TGFBI): c.593C > T (p.Ser198Phe) | |
| NM_000358.2(TGFBI): c.1998G > C (p.Arg666Ser) | hs101535656, hs101535663, |
| | hs101535663, hs101535656 |
| NM_000358.2(TGFBI): c.1526T > G (p.Leu509Arg) | hs101534814, hs101534815, |
| | hs101534824 |
| NM_000358.2(TGFBI): c.1619T > C (p.Phe540Ser) | hs101534957 |
| NM_000358.2(TGFBI): c.370C > A (p.Arg124Ser) | hs101533619, hs101533615, |
| | hs101533616, hs101533625 |
| TGFBI, 3-BP DEL | |
| NM_000358.2(TGFBI): c.1868G > A (p.Gly623Asp) | hs101535371, hs101535375, |
| | hs101535372, hs101535376 |
| NM_000358.2(TGFBI): c.371G > A (p.Arg124His) | hs101533619, hs101533615, |
| | hs101533616, hs101533625 |
| NM_000358.2(TGFBI): c.370C > T (p.Arg124Cys) | hs101533619, hs101533615, |
| | hs101533616, hs101533625 |
| NM_000358.2(TGFBI): c.1663C > T (p.Arg555Trp) | hs101534964, hs101534958, |
| | hs101534965, hs101534966, |
| | hs101534967, hs101534968, |
| | hs101534961, hs101534962 |
| NM_000358.2(TGFBI): c.[1637C > A;1652C > A] | hs101534959, hs101534960 |
| NM_000358.2(TGFBI): c.371G > T (p.Arg124Leu) | hs101533619, hs101533615, |
| | hs101533616, hs101533625 |
| NM_000358.2(TGFBI): c.1664G > A (p.Arg555Gln) | hs101534964, hs101534958, |
| | hs101534965, hs101534966, |
| | hs101534967, hs101534968, |
| | hs101534961, hs101534962 |
| NM_000358.2(TGFBI): c.1501C > A (p.Pro501Thr) | hs101534807, hs101534813, |
| | hs101534808, hs101534809, |
| | hs101534816, hs101534812, |
| | hs101534817, hs101534818, |
| | hs101534819, hs101534820, |
| | hs101534821 |
| NM_013319.2(UBIAD1): c.530G > A (p.Gly177Glu) | hs001050141, hs001050144, |
| | hs001050142 & hs001050500, |
| | hs001050502, hs001050507, |
| | hs001050504 |
| NM_013319.2(UBIAD1): c.708C > G (p.Asp236Glu) | hs001050535, hs001050534, |
| | hs001050538, hs001050546, |
| | hs001050540, hs001050547, |
| | hs001050542, hs001050543 |
| NM_013319.2(UBIAD1): c.335A > G (p.Asp112Gly) | hs001050116 |
| NM_013319.2(UBIAD1): c.355A > G (p.Arg119Gly) | hs001050115, hs001050119, |
| | hs001050117 |
| NM_013319.2(UBIAD1): c.556G > A (p.Gly186Arg) | hs001050505, hs001050506 |
| NM_013319.2(UBIAD1): c.511T > C (p.Ser171Pro) | hs001050143, hs001050140, |
| | hs001050141 |
| NM_013319.2(UBIAD1): c.695A > G (p.Asn232Ser) | hs001050539, hs001050541, |
| | hs001050534, hs001050535, |
| | hs001050544, hs001050538, |
| | hs001050546 |
| NM_013319.2(UBIAD1): c.524C > T (p.Thr175Ile) | hs001050143, hs001050140, |
| | hs001050141, hs001050144, |
| | hs001050142 |
| NM_013319.2(UBIAD1): c.529G > C (p.Gly177Arg) | hs001050141, hs001050144, |
| | hs001050142 & hs001050500, |
| | hs001050502, hs001050507, |
| | hs001050504 |
| NM_013319.2(UBIAD1): c.305A > G (p.Asn102Ser) | hs001050114 |
| NM_030751.5(ZEB1): c.2519A > C (p.Gln840Pro) | hs013097041, hs013097042, |
| | hs013097045, hs013097046 |
| NM_030751.5(ZEB1): c.233A > C (p.Asn78Thr) | hs013095774, hs013095775, |
| | hs013095776 |

TABLE 2
The gRNA target sequences by ID in Table 1 and corresponding
human genomic sequence.

| # | ID | Genomic Sequence |
|---|---|---|
| 1 | hs001050117 | GAGTGATGACAGGACACTTGTGG (SEQ ID NO: 1) |
| 2 | hs001050505 | TGGATTCAAGTACGTGGCTCTGG (SEQ ID NO: 2) |
| 3 | hs001050119 | TCCTGTCATCACTCTTTTTGTGG (SEQ ID NO: 3) |
| 4 | hs001050140 | TCTGGCTCCTTTCTCTACACAGG (SEQ ID NO: 4) |
| 5 | hs001050141 | GGCTCCTTTCTCTACACAGGAGG (SEQ ID NO: 5) |
| 6 | hs001050141 | GGCTCCTTTCTCTACACAGGAGG (SEQ ID NO: 6) |
| 7 | hs001050539 | GTTGGAATGGAGAATGGCCTCGG (SEQ ID NO: 7) |
| 8 | hs001050142 | TCTACACAGGAGGTAAGATTTGG (SEQ ID NO: 8) |
| 9 | hs001050142 | TCTACACAGGAGGTAAGATTTGG (SEQ ID NO: 9) |
| 10 | hs001050141 | GGCTCCTTTCTCTACACAGGAGG (SEQ ID NO: 10) |
| 11 | hs001050144 | CTTACCTCCTGTGTAGAGAAAGG (SEQ ID NO: 11) |
| 12 | hs001050500 | TCTCTGGATTTTCTGGCCGCAGG (SEQ ID NO: 12) |
| 13 | hs001050502 | GATTTTCTGGCCGCAGGAATTGG (SEQ ID NO: 13) |
| 14 | hs001050504 | AGGAATTGGATTCAAGTACGTGG (SEQ ID NO: 14) |
| 15 | hs001050114 | GTAAGTGTTGACCAAATTACCGG (SEQ ID NO: 15) |
| 16 | hs028999998 | ACCCGAATAAGAAGCCTTTTTGG (SEQ ID NO: 16) |
| 17 | hs001050504 | AGGAATTGGATTCAAGTACGTGG (SEQ ID NO: 17) |
| 18 | hs001050535 | TCTCCATTCCAACAACACCAGGG (SEQ ID NO: 18) |
| 19 | hs001050506 | GGATTCAAGTACGTGGCTCTGGG (SEQ ID NO: 19) |
| 20 | hs001050143 | TGTAGAGAAAGGAGCCAGACAGG (SEQ ID NO: 20) |
| 21 | hs001050507 | GTACTTGAATCCAATTCCTGCGG (SEQ ID NO: 21) |
| 22 | hs001050534 | TTCTCCATTCCAACAACACCAGG (SEQ ID NO: 22) |
| 23 | hs001050535 | TCTCCATTCCAACAACACCAGGG (SEQ ID NO: 23) |
| 24 | hs001050538 | TTCCAACAACACCAGGGACATGG (SEQ ID NO: 24) |
| 25 | hs001050540 | CCAGGGACATGGAGTCCGACCGG (SEQ ID NO: 25) |
| 26 | hs001050541 | GGTGTTGTTGGAATGGAGAATGG (SEQ ID NO: 26) |
| 27 | hs001050542 | CAGGGACATGGAGTCCGACCGGG (SEQ ID NO: 27) |
| 28 | hs001050543 | GGACATGGAGTCCGACCGGGAGG (SEQ ID NO: 28) |
| 29 | hs001050116 | CTTTTTGTGGTCAATGCCCTTGG (SEQ ID NO: 29) |
| 30 | hs001050115 | ACCACAAAAAGAGTGATGACAGG (SEQ ID NO: 30) |
| 31 | hs001050544 | TGTCCCTGGTGTTGTTGGAATGG (SEQ ID NO: 31) |
| 32 | hs001050546 | CTCCATGTCCCTGGTGTTGTTGG (SEQ ID NO: 32) |
| 33 | hs001050546 | CTCCATGTCCCTGGTGTTGTTGG (SEQ ID NO: 33) |
| 34 | hs001050143 | TGTAGAGAAAGGAGCCAGACAGG (SEQ ID NO: 34) |
| 35 | hs001050547 | CCGGTCGGACTCCATGTCCCTGG (SEQ ID NO: 35) |
| 36 | hs002677505 | CCAAGTCACCTTTCTGCCCCAGG (SEQ ID NO: 36) |
| 37 | hs002677506 | CAAGTCACCTTTCTGCCCCAGGG (SEQ ID NO: 37) |
| 38 | hs002677506 | CAAGTCACCTTTCTGCCCCAGGG (SEQ ID NO: 38) |
| 39 | hs013097041 | GCAAACGATTCTGATTCCCCAGG (SEQ ID NO: 39) |
| 40 | hs002677508 | AAGGTGACTTGGGGCTCCCTGGG (SEQ ID NO: 40) |
| 41 | hs002677509 | AAAGGTGACTTGGGGCTCCCTGG (SEQ ID NO: 41) |
| 42 | hs002677510 | CCCAGGGCTCCTGCCACCCCTGG (SEQ ID NO: 42) |
| 43 | hs002677511 | TGGGGCAGAAAGGTGACTTGGGG (SEQ ID NO: 43) |
| 44 | hs002677512 | CTGGGGCAGAAAGGTGACTTGGG (SEQ ID NO: 44) |
| 45 | hs002677513 | CCTGGGGCAGAAAGGTGACTTGG (SEQ ID NO: 45) |
| 46 | hs002677516 | GCAGGAGCCCTGGGGCAGAAAGG (SEQ ID NO: 46) |
| 47 | hs002677517 | CAGGGGTGGCAGGAGCCCTGGGG (SEQ ID NO: 47) |
| 48 | hs002677518 | CCAGGGGTGGCAGGAGCCCTGGG (SEQ ID NO: 48) |
| 49 | hs002677507 | TTGGGGCTCCCTGGGCAGCCTGG (SEQ ID NO: 49) |
| 50 | hs013095775 | AGGGAATGCTAAGAACTGCTGGG (SEQ ID NO: 50) |
| 51 | hs013095776 | GAATGCTAAGAACTGCTGGGAGG (SEQ ID NO: 51) |
| 52 | hs013097042 | AACGATTCTGATTCCCCAGGTGG (SEQ ID NO: 52) |
| 53 | hs013097045 | GAGTAGGTGTATGCCACCTGGGG (SEQ ID NO: 53) |
| 54 | hs013097046 | TGAGTAGGTGTATGCCACCTGGG (SEQ ID NO: 54) |
| 55 | hs013095774 | AAGGGAATGCTAAGAACTGCTGG (SEQ ID NO: 55) |
| 56 | hs027503293 | CTCTCCATGCTGCTCGGCCTCGG (SEQ ID NO: 56) |
| 57 | hs027503294 | ATGCTGCTCGGCCTCGGCAATGG (SEQ ID NO: 57) |
| 58 | hs027503297 | GGCCGAGCAGCATGGAGAGATGG (SEQ ID NO: 58) |
| 59 | hs027503299 | ATTGCCGAGGCCGAGCAGCATGG (SEQ ID NO: 59) |
| 60 | hs027503911 | CTCCGCCAGCTCCCCCAGAGTGG (SEQ ID NO: 60) |
| 61 | hs027503912 | TCCGCCAGCTCCCCCAGAGTGGG (SEQ ID NO: 61) |
| 62 | hs027503914 | GGAGCTGGCGGAGGGGCCTATGG (SEQ ID NO: 62) |
| 63 | hs027503915 | CTCTGGGGGAGCTGGCGGAGGGG (SEQ ID NO: 63) |
| 64 | hs027503916 | ACTCTGGGGGAGCTGGCGGAGGG (SEQ ID NO: 64) |
| 65 | hs027503917 | CACTCTGGGGGAGCTGGCGGAGG (SEQ ID NO: 65) |
| 66 | hs027503918 | GCCCACTCTGGGGGAGCTGGCGG (SEQ ID NO: 66) |
| 67 | hs027503919 | GTGGCCCACTCTGGGGGAGCTGG (SEQ ID NO: 67) |
| 68 | hs027503921 | AGCTGTGTGGCCCACTCTGGGGG (SEQ ID NO: 68) |
| 69 | hs027503292 | GGCCATCTCTCCATGCTGCTCGG (SEQ ID NO: 69) |
| 70 | hs028999999 | GCCTTTTTGGTGTTGTGTCCAGG (SEQ ID NO: 70) |
| 71 | hs029000000 | TTTTTGGTGTTGTGTCCAGGTGG (SEQ ID NO: 71) |
| 72 | hs029000000 | TTTTTGGTGTTGTGTCCAGGTGG (SEQ ID NO: 72) |
| 73 | hs028999999 | GCCTTTTTGGTGTTGTGTCCAGG (SEQ ID NO: 73) |
| 74 | hs029000001 | TTTTGGTGTTGTGTCCAGGTGGG (SEQ ID NO: 74) |
| 75 | hs029000002 | ACCAAAAAGGCTTCTTATTCGGG (SEQ ID NO: 75) |
| 76 | hs029000003 | CACCAAAAAGGCTTCTTATTCGG (SEQ ID NO: 76) |
| 77 | hs029000003 | CACCAAAAAGGCTTCTTATTCGG (SEQ ID NO: 77) |
| 78 | hs029000000 | TTTTTGGTGTTGTGTCCAGGTGG (SEQ ID NO: 78) |
| 79 | hs029000004 | ACCTGGACACAACACCAAAAAGG (SEQ ID NO: 79) |
| 80 | hs029000005 | TCAAGTGACTTCTGCCCACCTGG (SEQ ID NO: 80) |
| 81 | hs028999998 | ACCCGAATAAGAAGCCTTTTTGG (SEQ ID NO: 81) |
| 82 | hs029000005 | TCAAGTGACTTCTGCCCACCTGG (SEQ ID NO: 82) |
| 83 | hs070544033 | GAGTTCACTGAGTCACTGTCGGG (SEQ ID NO: 83) |
| 84 | hs051020748 | CTTGGGCCTCCCCGTCCAGCAGG (SEQ ID NO: 84) |
| 85 | hs051020750 | GGGCCTCCCCGTCCAGCAGGCGG (SEQ ID NO: 85) |
| 86 | hs051020752 | CCTCCCCGTCCAGCAGGCGGCGG (SEQ ID NO: 86) |
| 87 | hs051020753 | CCCGTCCAGCAGGCGGCGGTAGG (SEQ ID NO: 87) |
| 88 | hs051020755 | CTGCTGGACGGGGAGGCCCAAGG (SEQ ID NO: 88) |
| 89 | hs051020756 | CCGCCGCCTGCTGGACGGGGAGG (SEQ ID NO: 89) |
| 90 | hs051020758 | CTACCGCCGCCTGCTGGACGGGG (SEQ ID NO: 90) |
| 91 | hs051020759 | CCTACCGCCGCCTGCTGGACGGG (SEQ ID NO: 91) |
| 92 | hs051020760 | ACCTACCGCCGCCTGCTGGACGG (SEQ ID NO: 92) |
| 93 | hs051020761 | TCTCAATCTCCAGCTCCAGGCGG (SEQ ID NO: 93) |
| 94 | hs051020762 | CTCAATCTCCAGCTCCAGGCGGG (SEQ ID NO: 94) |
| 95 | hs051020757 | AGGTCTCAATCTCCAGCTCCAGG (SEQ ID NO: 95) |
| 96 | hs051020762 | CTCAATCTCCAGCTCCAGGCGGG (SEQ ID NO: 96) |
| 97 | hs051021069 | CTAGAGCTCGCACCTTATCCAGG (SEQ ID NO: 97) |
| 98 | hs051020763 | TGAGACCTACCGCCGCCTGCTGG (SEQ ID NO: 98) |
| 99 | hs051020752 | CCTCCCCGTCCAGCAGGCGGCGG (SEQ ID NO: 99) |
| 100 | hs051020763 | TGAGACCTACCGCCGCCTGCTGG (SEQ ID NO: 100) |
| 101 | hs051020757 | AGGTCTCAATCTCCAGCTCCAGG (SEQ ID NO: 101) |
| 102 | hs051020763 | TGAGACCTACCGCCGCCTGCTGG (SEQ ID NO: 102) |
| 103 | hs051020758 | CTACCGCCGCCTGCTGGACGGGG (SEQ ID NO: 103) |
| 104 | hs051020763 | TGAGACCTACCGCCGCCTGCTGG (SEQ ID NO: 104) |
| 105 | hs051021073 | TGATAGATTAGCTTCCTACCTGG (SEQ ID NO: 105) |
| 106 | hs051021073 | TGATAGATTAGCTTCCTACCTGG (SEQ ID NO: 106) |
| 107 | hs051021071 | TAAGGTGCGAGCTCTAGAAGAGG (SEQ ID NO: 107) |
| 108 | hs051021069 | CTAGAGCTCGCACCTTATCCAGG (SEQ ID NO: 108) |
| 109 | hs051021070 | AGCTCGCACCTTATCCAGGTAGG (SEQ ID NO: 109) |
| 110 | hs051021070 | AGCTCGCACCTTATCCAGGTAGG (SEQ ID NO: 110) |
| 111 | hs051021072 | ATTAGCTTCCTACCTGGATAAGG (SEQ ID NO: 111) |
| 112 | hs051021072 | ATTAGCTTCCTACCTGGATAAGG (SEQ ID NO: 112) |
| 113 | hs051021073 | TGATAGATTAGCTTCCTACCTGG (SEQ ID NO: 113) |
| 114 | hs051020758 | CTACCGCCGCCTGCTGGACGGGG (SEQ ID NO: 114) |
| 115 | hs051021073 | TGATAGATTAGCTTCCTACCTGG (SEQ ID NO: 115) |
| 116 | hs051021073 | TGATAGATTAGCTTCCTACCTGG (SEQ ID NO: 116) |
| 117 | hs051020754 | TGCTGGACGGGGAGGCCCAAGGG (SEQ ID NO: 117) |
| 118 | hs051021143 | CACTCTGCGAGGAGAGCCGCCGG (SEQ ID NO: 118) |
| 119 | hs051021144 | ACTCTGCGAGGAGAGCCGCCGGG (SEQ ID NO: 119) |
| 120 | hs051021148 | GGAGAGCCGCCGGGACAGTCCGG (SEQ ID NO: 120) |
| 121 | hs051021149 | GAGAGCCGCCGGGACAGTCCGGG (SEQ ID NO: 121) |
| 122 | hs051021150 | AGAGCCGCCGGGACAGTCCGGGG (SEQ ID NO: 122) |
| 123 | hs051021151 | GAGCCGCCGGGACAGTCCGGGGG (SEQ ID NO: 123) |
| 124 | hs051021154 | GCACCCCCGGACTGTCCCGGCGG (SEQ ID NO: 124) |
| 125 | hs051021155 | TGCGCACCCCCGGACTGTCCCGG (SEQ ID NO: 125) |
| 126 | hs051021155 | TGCGCACCCCCGGACTGTCCCGG (SEQ ID NO: 126) |
| 127 | hs051021148 | GGAGAGCCGCCGGGACAGTCCGG (SEQ ID NO: 127) |
| 128 | hs051021157 | TCACTCTCAGTGCGCACCCCCGG (SEQ ID NO: 128) |
| 129 | hs051021071 | TAAGGTGCGAGCTCTAGAAGAGG (SEQ ID NO: 129) |
| 130 | hs070544030 | CAGTGACTCAGTGAACTCCGTGG (SEQ ID NO: 130) |
| 131 | hs070544031 | GACTCAGTGAACTCCGTGGAAGG (SEQ ID NO: 131) |
| 132 | hs070545604 | GCAAAAGCAATGATGGAGCTGGG (SEQ ID NO: 132) |
| 133 | hs070544034 | GGAGTTCACTGAGTCACTGTCGG (SEQ ID NO: 133) |
| 134 | hs070544847 | GATGACCAACAAGATGCTTTAGG (SEQ ID NO: 134) |
| 135 | hs070544849 | CATCTTGTTGGTCATCCACAGGG (SEQ ID NO: 135) |
| 136 | hs070544850 | GCATCTTGTTGGTCATCCACAGG (SEQ ID NO: 136) |
| 137 | hs070544851 | CGCTGCCTAAAGCATCTTGTTGG (SEQ ID NO: 137) |
| 138 | hs070544887 | GGAGGAAGAAACAGCTGCTCAGG (SEQ ID NO: 138) |
| 139 | hs070544888 | GAGGAAGAAACAGCTGCTCAGGG (SEQ ID NO: 139) |
| 140 | hs131603026 | ATGGAAGAGAAACTCATGATCGG (SEQ ID NO: 140) |
| 141 | hs070545601 | CCATCATTGCTTTTGCTCTCAGG (SEQ ID NO: 141) |
| 142 | hs070545605 | AGCAAAAGCAATGATGGAGCTGG (SEQ ID NO: 142) |
| 143 | hs070545606 | CCTGAGAGCAAAAGCAATGATGG (SEQ ID NO: 143) |
| 144 | hs070544848 | TGTTGGTCATCCACAGGGAGTGG (SEQ ID NO: 144) |
| 145 | hs101533615 | TCAGCTGTACACGGACCGCACGG (SEQ ID NO: 145) |
| 146 | hs101533616 | CGGACCGCACGGAGAAGCTGAGG (SEQ ID NO: 146) |
| 147 | hs101533625 | CAGGCCTCAGCTTCTCCGTGCGG (SEQ ID NO: 147) |
| 148 | hs101533619 | GCGGTCCGTGTACAGCTGAGTGG (SEQ ID NO: 148) |
| 149 | hs101533625 | CAGGCCTCAGCTTCTCCGTGCGG (SEQ ID NO: 149) |
| 150 | hs101534964 | GTTCTCTTGGTGGCAGGGCTCGG (SEQ ID NO: 150) |
| 151 | hs101533625 | CAGGCCTCAGCTTCTCCGTGCGG (SEQ ID NO: 151) |
| 152 | hs101535371 | CCTGACATCATGGCCACAAATGG (SEQ ID NO: 152) |
| 153 | hs101534808 | CGGGTGCTGACCCCCCCAATGGG (SEQ ID NO: 153) |
| 154 | hs101534809 | GGGTGCTGACCCCCCCAATGGGG (SEQ ID NO: 154) |
| 155 | hs101534812 | CCCCCCAATGGGGACTGTCATGG (SEQ ID NO: 155) |
| 156 | hs101534813 | CCATTGGGGGGGTCAGCACCCGG (SEQ ID NO: 156) |
| 157 | hs101534815 | ACTGTCATGGATGTCCTGAAGGG (SEQ ID NO: 157) |
| 158 | hs101534816 | CATGACAGTCCCCATTGGGGGGG (SEQ ID NO: 158) |
| 159 | hs101534817 | CCATGACAGTCCCCATTGGGGGG (SEQ ID NO: 159) |
| 160 | hs101534818 | TCCATGACAGTCCCCATTGGGGG (SEQ ID NO: 160) |
| 161 | hs101534819 | ATCCATGACAGTCCCCATTGGGG (SEQ ID NO: 161) |
| 162 | hs101534820 | CATCCATGACAGTCCCCATTGGG (SEQ ID NO: 162) |
| 163 | hs101534821 | ACATCCATGACAGTCCCCATTGG (SEQ ID NO: 163) |
| 164 | hs101534824 | TAAAGCGATTGTCTCCCTTCAGG (SEQ ID NO: 164) |
| 165 | hs101534957 | AGACTGTGTAGACTCCTTCCCGG (SEQ ID NO: 165) |
| 166 | hs101533619 | GCGGTCCGTGTACAGCTGAGTGG (SEQ ID NO: 166) |
| 167 | hs101534958 | GAGCCCTGCCACCAAGAGAACGG (SEQ ID NO: 167) |
| 168 | hs101534960 | GGCTCGGAAGGCTTCATTTGTGG (SEQ ID NO: 168) |
| 169 | hs101533619 | GCGGTCCGTGTACAGCTGAGTGG (SEQ ID NO: 169) |
| 170 | hs101534961 | AAGAGAACGGAGCAGACTCTTGG (SEQ ID NO: 170) |
| 171 | hs101534962 | AGAGAACGGAGCAGACTCTTGGG (SEQ ID NO: 171) |
| 172 | hs101534807 | CCGGGTGCTGACCCCCCCAATGG (SEQ ID NO: 172) |
| 173 | hs101534962 | AGAGAACGGAGCAGACTCTTGGG (SEQ ID NO: 171) |
| 174 | hs101534959 | GCTCGGAAGGCTTCATTTGTGGG (SEQ ID NO: 174) |
| 175 | hs101534965 | GCTCCGTTCTCTTGGTGGCAGGG (SEQ ID NO: 175) |
| 176 | hs101534966 | TGCTCCGTTCTCTTGGTGGCAGG (SEQ ID NO: 176) |
| 177 | hs101534967 | AGTCTGCTCCGTTCTCTTGGTGG (SEQ ID NO: 177) |
| 178 | hs101534968 | AAGAGTCTGCTCCGTTCTCTTGG (SEQ ID NO: 178) |
| 179 | hs101535372 | CATCATGGCCACAAATGGCGTGG (SEQ ID NO: 179) |
| 180 | hs101535375 | CCATTTGTGGCCATGATGTCAGG (SEQ ID NO: 180) |
| 181 | hs101535376 | GACATGGACCACGCCATTTGTGG (SEQ ID NO: 181) |
| 182 | hs101533619 | GCGGTCCGTGTACAGCTGAGTGG (SEQ ID NO: 182) |
| 183 | hs101535656 | TTTTCTTTCAGGCTTCCCAGAGG (SEQ ID NO: 183) |
| 184 | hs101535656 | TTTTCTTTCAGGCTTCCCAGAGG (SEQ ID NO: 184) |
| 185 | hs101534814 | GACTGTCATGGATGTCCTGAAGG (SEQ ID NO: 185) |
| 186 | hs101535663 | ACCTAGTCGCACAGACCTCTGGG (SEQ ID NO: 186) |
| 187 | hs131602459 | AGGAGGAAAAGCAAACAACTGGG (SEQ ID NO: 187) |
| 188 | hs131602461 | GGAAAAGCAAACAACTGGGAAGG (SEQ ID NO: 188) |
| 189 | hs131602462 | AAAGCAAACAACTGGGAAGGAGG (SEQ ID NO: 189) |
| 190 | hs131602463 | ACAACTGGGAAGGAGGTATCCGG (SEQ ID NO: 190) |
| 191 | hs131602464 | CAACTGGGAAGGAGGTATCCGGG (SEQ ID NO: 191) |
| 192 | hs002677511 | TGGGGCAGAAAGGTGACTTGGGG (SEQ ID NO: 192) |
| 193 | hs131603028 | TTAAGTAGGCGTTGCAGTAATGG (SEQ ID NO: 193) |
| 194 | hs131602458 | TAGGAGGAAAAGCAAACAACTGG (SEQ ID NO: 194) |

TABLE 3
The gRNA target sequences by ID and human genomic sequence
in the TCF4 gene upstream of the microsatellite expansion
causing Fuchs endothelial corneal dystrophy.

| # | ID | Genomic Sequence |
|---|---|---|
| 1 | hs056193542 | AAAGAGCCCCACTTGGAAGGCGG (SEQ ID NO: 195) |
| 2 | hs056193543 | GCCCCACTTGGAAGGCGGTTTGG (SEQ ID NO: 196) |
| 3 | hs056193544 | GATTTTATTTGTGTGTTTTGTGG (SEQ ID NO: 197) |
| 4 | hs056193545 | TCCAAACCGCCTTCCAAGTGGGG (SEQ ID NO: 198) |
| 5 | hs056193546 | ATCCAAACCGCCTTCCAAGTGGG (SEQ ID NO: 199) |
| 6 | hs056193547 | AATCCAAACCGCCTTCCAAGTGG (SEQ ID NO: 200) |
| 7 | hs056193548 | CATCTTACACCAAACTCATCTGG (SEQ ID NO: 201) |
| 8 | hs056193549 | TTTTTAATGCCAGATGAGTTTGG (SEQ ID NO: 202) |
| 9 | hs056193550 | ATTCATTCTCCTGACATGTCTGG (SEQ ID NO: 203) |
| 10 | hs056193551 | TTCATTCTCCTGACATGTCTGGG (SEQ ID NO: 204) |
| 11 | hs056193552 | CTCCTGACATGTCTGGGACTTGG (SEQ ID NO: 205) |
| 12 | hs056193553 | ACATGTCTGGGACTTGGTTTAGG (SEQ ID NO: 206) |
| 13 | hs056193554 | CTGGGACTTGGTTTAGGAAAAGG (SEQ ID NO: 207) |
| 14 | hs056193555 | GGTTTAGGAAAAGGAAGCAAAGG (SEQ ID NO: 208) |
| 15 | hs056193556 | GTTTAGGAAAAGGAAGCAAAGGG (SEQ ID NO: 209) |
| 16 | hs056193557 | AACCAAGTCCCAGACATGTCAGG (SEQ ID NO: 210) |
| 17 | hs056193558 | AGGAAAAGGAAGCAAAGGGATGG (SEQ ID NO: 211) |
| 18 | hs056193559 | AGGAAGCAAAGGGATGGAGAAGG (SEQ ID NO: 212) |
| 19 | hs056193560 | TGGAGTTTTACGGCTGTACTTGG (SEQ ID NO: 213) |
| 20 | hs056193561 | GACACACTTGTGGAGTTTTACGG (SEQ ID NO: 214) |
| 21 | hs056193562 | AGCGGAACTTGACACACTTGTGG (SEQ ID NO: 215) |
| 22 | hs056193563 | GTCGTAGGATCAGCACAAAGCGG (SEQ ID NO: 216) |
| 23 | hs056193564 | ATTTACCAAAACAGTCCAAAAGG (SEQ ID NO: 217) |
| 24 | hs056193565 | TTGGTAAATTTCGTAGTCGTAGG (SEQ ID NO: 218) |
| 25 | hs056193566 | TAGAACCTTTTGGACTGTTTTGG (SEQ ID NO: 219) |
| 26 | hs056193567 | ATACATTCTTTAGAACCTTTTGG (SEQ ID NO: 220) |
| 27 | hs056193568 | ATACTAGTTTTAAGAATCCTAGG (SEQ ID NO: 221) |
| 28 | hs056193569 | TCCTAGGAAAAGATGTAACTAGG (SEQ ID NO: 222) |
| 29 | hs056193570 | TAGGAAAAGATGTAACTAGGAGG (SEQ ID NO: 223) |
| 30 | hs056193571 | TAGGATTCTTAAAACTAGTATGG (SEQ ID NO: 224) |
| 31 | hs056193572 | TAACTAGGAGGTAAGATGTAAGG (SEQ ID NO: 225) |
| 32 | hs056193573 | GGAGGTAAGATGTAAGGAACAGG (SEQ ID NO: 226) |
| 33 | hs056193574 | TCCTAGTTACATCTTTTCCTAGG (SEQ ID NO: 227) |
| 34 | hs056193575 | TAATGATGCTTTGGATTGGTAGG (SEQ ID NO: 228) |
| 35 | hs056193576 | AAGCTAATGATGCTTTGGATTGG (SEQ ID NO: 229) |
| 36 | hs056193577 | TAAAACTTTAAAGAGACAACTGG (SEQ ID NO: 230) |
| 37 | hs056193578 | AAAACTTTAAAGAGACAACTGGG (SEQ ID NO: 231) |
| 38 | hs056193579 | GTTTTAAGCTAATGATGCTTTGG (SEQ ID NO: 232) |
| 39 | hs056193580 | GGAAATGGAAAATAGAAAATAGG (SEQ ID NO: 233) |
| 40 | hs056193581 | TTATTTATTGTTTTTGGAAATGG (SEQ ID NO: 234) |
| 41 | hs056193582 | TTCGTTTTATTTATTGTTTTTGG (SEQ ID NO: 235) |
| 42 | hs056193583 | GTAGTCTCAGTGTTCAGACATGG (SEQ ID NO: 236) |
| 43 | hs056193584 | TTCAGACATGGCCAAGTTTTAGG (SEQ ID NO: 237) |
| 44 | hs056193585 | TCAGACATGGCCAAGTTTTAGGG (SEQ ID NO: 238) |
| 45 | hs056193586 | CAGACATGGCCAAGTTTTAGGGG (SEQ ID NO: 239) |
| 46 | hs056193587 | ACATGGCCAAGTTTTAGGGGTGG (SEQ ID NO: 240) |
| 47 | hs056193588 | TTTAGGGGTGGTTTAGTTTTAGG (SEQ ID NO: 241) |
| 48 | hs056193589 | TTAGGGGTGGTTTAGTTTTAGGG (SEQ ID NO: 242) |
| 49 | hs056193590 | TAGGGGTGGTTTAGTTTTAGGGG (SEQ ID NO: 243) |
| 50 | hs056193591 | ACTAAACCACCCCTAAAACTTGG (SEQ ID NO: 244) |
| 51 | hs056193592 | TGTCTATTTTTGCTTTCCACTGG (SEQ ID NO: 245) |
| 52 | hs056193593 | GTCTATTTTTGCTTTCCACTGGG (SEQ ID NO: 246) |
| 53 | hs056193594 | TCTATTTTTGCTTTCCACTGGGG (SEQ ID NO: 247) |
| 54 | hs056193595 | TGGGGTGAGATTCCATTATTTGG (SEQ ID NO: 248) |
| 55 | hs056193596 | GGGGTGAGATTCCATTATTTGGG (SEQ ID NO: 249) |
| 56 | hs056193597 | GGGTGAGATTCCATTATTTGGGG (SEQ ID NO: 250) |
| 57 | hs056193598 | CCATTATTTGGGGTAATCAGTGG (SEQ ID NO: 251) |
| 58 | hs056193599 | CATTATTTGGGGTAATCAGTGGG (SEQ ID NO: 252) |
| 59 | hs056193600 | ATTTGGGGTAATCAGTGGGTAGG (SEQ ID NO: 253) |
| 60 | hs056193601 | ATAATGGAATCTCACCCCAGTGG (SEQ ID NO: 254) |
| 61 | hs056193602 | TTTGGGGTAATCAGTGGGTAGGG (SEQ ID NO: 255) |
| 62 | hs056193603 | ATCAGTGGGTAGGGAATTGAAGG (SEQ ID NO: 256) |
| 63 | hs056193604 | CCACTGATTACCCCAAATAATGG (SEQ ID NO: 257) |
| 64 | hs056193605 | TTTTTTTTGAGTTTTATTACTGG (SEQ ID NO: 258) |
| 65 | hs056193606 | TGTGGTGTGATGGAAGATTCAGG (SEQ ID NO: 259) |
| 66 | hs056193607 | ACTATAATTTTGTGGTGTGATGG (SEQ ID NO: 260) |
| 67 | hs056193608 | AGTTTTTAACTATAATTTTGTGG (SEQ ID NO: 261) |
| 68 | hs056193609 | AAAGACCTTCATATTTACCAAGG (SEQ ID NO: 262) |
| 69 | hs056193610 | TGAATCCTTGGTAAATATGAAGG (SEQ ID NO: 263) |
| 70 | hs056193611 | TTTTTAATTGGCTGAATCCTTGG (SEQ ID NO: 264) |
| 71 | hs056193612 | ACTGTCCTTTAGATTCCTACTGG (SEQ ID NO: 265) |
| 72 | hs056193613 | GGACAGTAATAATTTTTAATTGG (SEQ ID NO: 266) |
| 73 | hs056193614 | TGGTTTCTAGCTGAAGTGTTTGG (SEQ ID NO: 267) |
| 74 | hs056193615 | GGTTTCTAGCTGAAGTGTTTGGG (SEQ ID NO: 268) |
| 75 | hs056193616 | AGAAACCAGTAGGAATCTAAAGG (SEQ ID NO: 269) |
| 76 | hs056193617 | CACTTCAGCTAGAAACCAGTAGG (SEQ ID NO: 270) |
| 77 | hs056193618 | AGTGCGGTAAGAAAGAACGGTGG (SEQ ID NO: 271) |
| 78 | hs056193619 | TTCAGTGCGGTAAGAAAGAACGG (SEQ ID NO: 272) |
| 79 | hs056193620 | TGATTTACTGGATTTCAGTGCGG (SEQ ID NO: 273) |

TABLE 4
The gRNA target sequences by ID and human genomic sequence
in the TCF4 gene downstream of the microsatellite expansion
causing Fuchs endothelial corneal dystrophy.

| # | ID | Genomic Sequence |
|---|---|---|
| 1 | hs056193473 | AGATCTTTGAGGAGCTCTGAAGG (SEQ ID NO: 274) |
| 2 | hs056193474 | AACAGTATGAAAGATCTTTGAGG (SEQ ID NO: 275) |
| 3 | hs056193475 | ACAGCTTAGAGTTTATGCTAAGG (SEQ ID NO: 276) |
| 4 | hs056193476 | CAGCTTAGAGTTTATGCTAAGGG (SEQ ID NO: 277) |
| 5 | hs056193477 | AGCATAAACTCTAAGCTGTTTGG (SEQ ID NO: 278) |
| 6 | hs056193478 | TCTTTTAGTTTTAAGTTGGATGG (SEQ ID NO: 279) |
| 7 | hs056193479 | TTTCTCTTTTAGTTTTAAGTTGG (SEQ ID NO: 280) |
| 8 | hs056193480 | GTGATAATGGGGGCTGGGGTGGG (SEQ ID NO: 281) |
| 9 | hs056193481 | AGTGATAATGGGGGCTGGGGTGG (SEQ ID NO: 282) |
| 10 | hs056193482 | TCTGTTCTTTCTTTTTCCTCAGG (SEQ ID NO: 283) |
| 11 | hs056193483 | CAGAGTGATAATGGGGGCTGGGG (SEQ ID NO: 284) |
| 12 | hs056193484 | ACAGAGTGATAATGGGGGCTGGG (SEQ ID NO: 285) |
| 13 | hs056193485 | AACAGAGTGATAATGGGGGCTGG (SEQ ID NO: 286) |
| 14 | hs056193486 | AAAGAACAGAGTGATAATGGGGG (SEQ ID NO: 287) |
| 15 | hs056193487 | GAAAGAACAGAGTGATAATGGGG (SEQ ID NO: 288) |
| 16 | hs056193488 | AGAAAGAACAGAGTGATAATGGG (SEQ ID NO: 289) |
| 17 | hs056193489 | AAGAAAGAACAGAGTGATAATGG (SEQ ID NO: 290) |
| 18 | hs056193490 | TTTTCCTCAGGTTCATTAGATGG (SEQ ID NO: 291) |
| 19 | hs056193491 | TTGGCCATCTAATGAACCTGAGG (SEQ ID NO: 292) |
| 20 | hs056193492 | AGCAGTACTACTGCTACATTTGG (SEQ ID NO: 293) |
| 21 | hs056193493 | AATGTAGCAGTAGTACTGCTTGG (SEQ ID NO: 294) |
| 22 | hs056193494 | CCATAATGTTATCAAGATTCAGG (SEQ ID NO: 295) |
| 23 | hs056193495 | AATGTTATCAAGATTCAGGTTGG (SEQ ID NO: 296) |
| 24 | hs056193496 | GTTATCAAGATTCAGGTTGGAGG (SEQ ID NO: 297) |
| 25 | hs056193497 | TGAATCTTGATAACATTATGGGG (SEQ ID NO: 298) |
| 26 | hs056193498 | CTGAATCTTGATAACATTATGGG (SEQ ID NO: 299) |
| 27 | hs056193499 | CCTGAATCTTGATAACATTATGG (SEQ ID NO: 300) |
| 28 | hs056193500 | GAAAAACACTAGTTTCACCAAGG (SEQ ID NO: 301) |
| 29 | hs056193501 | TGTTTTTCTAGAGAGGCTGCTGG (SEQ ID NO: 302) |
| 30 | hs056193502 | AAACTAGTGTTTTTCTAGAGAGG (SEQ ID NO: 303) |
| 31 | hs056193503 | AACAACTTTTTTCTTCTCCTTGG (SEQ ID NO: 304) |
| 32 | hs056193504 | TTGTTTTATATTGAAAACCTTGG (SEQ ID NO: 305) |
| 33 | hs056193505 | GAAAACCTTGGCCATAAACGTGG (SEQ ID NO: 306) |
| 34 | hs056193506 | TGTCCATTTCCATCTCGTATAGG (SEQ ID NO: 307) |
| 35 | hs056193507 | CATTGCCACGTTTATGGCCAAGG (SEQ ID NO: 308) |
| 36 | hs056193508 | AATGGACATTGCCACGTTTATGG (SEQ ID NO: 309) |
| 37 | hs056193509 | AATCCTATACGAGATGGAAATGG (SEQ ID NO: 310) |
| 38 | hs056193510 | CAGGCAAATCCTATACGAGATGG (SEQ ID NO: 311) |
| 39 | hs056193511 | TATTTGGGTTCACATATGACAGG (SEQ ID NO: 312) |
| 40 | hs056193512 | TGGCACTTTTATTTTTATTTGGG (SEQ ID NO: 313) |
| 41 | hs056193513 | GTGGCACTTTTATTTTTATTTGG (SEQ ID NO: 314) |
| 42 | hs056193514 | ATTCTCATTTCGTCTCTAACAGG (SEQ ID NO: 315) |
| 43 | hs056193515 | AAATGAGAATTTAGTGCAGGTGG (SEQ ID NO: 316) |
| 44 | hs056193516 | ACGAAATGAGAATTTAGTGCAGG (SEQ ID NO: 317) |
| 45 | hs056193517 | GCATTTATTTCGACCCTAATTGG (SEQ ID NO: 318) |
| 46 | hs056193518 | CTCTTCTTCGACGTATCTAGTGG (SEQ ID NO: 319) |
| 47 | hs056193519 | AAGAAGAGGGAAACCAATTAGGG (SEQ ID NO: 320) |
| 48 | hs056193520 | GAAGAAGAGGGAAACCAATTAGG (SEQ ID NO: 321) |
| 49 | hs056193521 | ACTAGATACGTCGAAGAAGAGGG (SEQ ID NO: 322) |
| 50 | hs056193522 | CACTAGATACGTCGAAGAAGAGG (SEQ ID NO: 323) |
| 51 | hs056193523 | TCAGAGCCTGCAAAAAGCAAAGG (SEQ ID NO: 324) |
| 52 | hs056193524 | GCAAAAAGCAAAGGAACGAATGG (SEQ ID NO: 325) |
| 53 | hs056193525 | TGCAGGCTCTGACTCAGGGAAGG (SEQ ID NO: 326) |
| 54 | hs056193526 | TTTTTGCAGGCTCTGACTCAGGG (SEQ ID NO: 327) |
| 55 | hs056193527 | CTTTTTGCAGGCTCTGACTCAGG (SEQ ID NO: 328) |
| 56 | hs056193528 | TTCGTTCCTTTGCTTTTTGCAGG (SEQ ID NO: 329) |
| 57 | hs056193529 | AGAAAGTGCAACAAGCAGAAAGG (SEQ ID NO: 330) |
| 58 | hs056193530 | GAAAGTGCAACAAGCAGAAAGGG (SEQ ID NO: 331) |
| 59 | hs056193531 | AAAGTGCAACAAGCAGAAAGGGG (SEQ ID NO: 332) |
| 60 | hs056193532 | AAGTGCAACAAGCAGAAAGGGGG (SEQ ID NO: 333) |
| 61 | hs056193533 | GGCTGCAAAGCTGCCTGCCTAGG (SEQ ID NO: 334) |
| 62 | hs056193534 | GCTGCAAAGCTGCCTGCCTAGGG (SEQ ID NO: 335) |
| 63 | hs056193535 | CTGCCTAGGGCTACGTTTCCTGG (SEQ ID NO: 336) |
| 64 | hs056193536 | CAGGAAACGTAGCCCTAGGCAGG (SEQ ID NO: 337) |
| 65 | hs056193537 | TTGCCAGGAAACGTAGCCCTAGG (SEQ ID NO: 338) |
| 66 | hs056193538 | AAAGCCATTTCTCCAAAAGAAGG (SEQ ID NO: 339) |
| 67 | hs056193539 | TGGCTTTCGGAAGTTTTGCCAGG (SEQ ID NO: 340) |
| 68 | hs056193540 | TCTTTTGGAGAAATGGCTTTCGG (SEQ ID NO: 341) |
| 69 | hs056193541 | TAGACCTTCTTTTGGAGAAATGG (SEQ ID NO: 342) |

TABLE 5
Microsatellite expansion diseases/conditions
with affected gene(s) in brackets.

Blepharophimosis, ptosis and epicanthus inversus syndactyly [FOXL2]
Cleidocranial dysplasia [RUNX2 CBFA1)]
Congenital central hypoventilation syndrome, Haddad syndrome
[PHOX2B]
DM (Myotonic dystrophy) [DMPK]
DRPLA (Dentatorubropallidoluysian atrophy) [ATN1 or DRPLA]
FRAXA (Fragile X syndrome) [FMR1]
FRAXE (Fragile XE mental retardation) [AFF2 or FMR2]
FRDA (Friedreich's ataxia) [FXN or X25]
Fuchs' Endothelial Corneal Dystrophy [TCF4]
FXTAS (Fragile X-associated tremor/ataxia syndrome) [FMR1]
Hand-foot-genital syndrome [HOXA13]
HD (Huntington's disease) [HTT (Huntingtin)]
Holoprosencephaly (HPE5) [ZIC2]
Mental retardation with growth hormone deficiency [SOX3]
Mental retardation, epilepsy, West syndrome, Partington syndrome [ARX]
Oculopharyngeal muscular dystrophy [PABPN1]
SBMA (Spinal and bulbar muscular atrophy) [AR]
SCA1 (Spinocerebellar ataxia Type 1) [ATXN1]
SCA12 (Spinocerebellar ataxia Type 12) [PPP2R2B or SCA12]
SCA17 (Spinocerebellar ataxia Type 17) [TBP]
SCA2 (Spinocerebellar ataxia Type 2) [ATXN2]
SCA3 (Spinocerebellar ataxia Type 3 or Machado-Joseph disease)
[ATXN3]
SCA6 (Spinocerebellar ataxia Type 6) [CACNA1A]
SCA7 (Spinocerebellar ataxia Type 7) [ATXN7]
SCA8 (Spinocerebellar ataxia Type 8) [OSCA or SCA8]
Synpolydactyly [HOXD13]

### REFERENCES

1. Weiss J S, Moller H U, Aldave A J, et al. IC3D classification of corneal dystrophies—edition 2. Cornea 2015; 34:117-159.
2. Afshari N A, Bouchard C S, Colby K A, et al. Corneal dystrophies and ectasias. In: Weisenthal R W, ed. 2014-2015 Basic and Clinical Science Course, Section 8: External Disease and Cornea. San Francisco; American Academy of Ophthalmology; 2014:253-287.
3. Weiss J S, Moller H U, Lisch W et al. The IC3D classification of the corneal dystrophies. Cornea 2008; 27(suppl2):S1-S83.
4. Wieben E D, Aleff R A, Eckloff B W, Atkinson E J, Baheti S, Middha S, et al. Comprehensive Assessment of Genetic Variants Within TCF4 in Fuchs' Endothelial Corneal Dystrophy. Investigative ophthalmology & visual science. 2014; 55(9):6101-7.
5. Nelson D L, Orr H T, Warren S T. The unstable repeats—three evolving faces of neurological disease. Neuron. 2013 Mar. 6; 77(5):825-43.
6. Hsu P D, Lander E S, Zhang F. Development and applications of CRISPR-Cas9 for genome engineering. Cell. 2014; 157(6):1262-78.
7. Doudna J A, Charpentier E. Genome editing. The new frontier of genome engineering with CRISPR-Cas9. Science. 2014; 346(6213):1258096.
8. Ranganathan V, Wahlin K, Maruotti J, Zack D J. Expansion of the CRISPR-Cas9 genome targeting space through the use of H1 promoter-expressed guide RNAs. Nature Communications. 2014; 5:4516.
9. Munier F L, Frueh B E, Othenin-Girard P, Uffer S, Cousin P, Wang M X, Heon E, Black G C M, Blasi M A, Balestrazzi E, Lorenz B, Escoto R, Barraquer R, Hoeltzenbein M, Gloor B, Fossarello M, Singh A D, Arsenijevic Y, Zografos L, Schorderet D F. BIGH3 mutation spectrum in corneal dystrophies. Invest. Ophthal. Vis. Sci. 43: 949-954, 2002.
10. Kleinstiver B P, Prew M S, Tsai S Q, Topkar V V, Nguyen N T, Zheng Z, Gonzales A P, Li Z, Peterson R T, Yeh J J, Aryee M J, Joung J K. Engineered CRISPR-Cas9 nucleases with altered PAM specificities. Nature. 2015 Jun. 22. doi: 10.1038/nature14592.

### EQUIVALENTS

Those skilled in the art will recognize, or be able to ascertain using no more than routine experimentation, many equivalents to the specific embodiments of the invention described herein. While specific embodiments of the subject invention have been discussed, the above specification is illustrative and not restrictive. Many variations of the invention may become apparent to those skilled in the art upon review of this specification. The full scope of the invention should be determined by reference to the claims, along with their full scope of equivalents, and the specification, along with such variations. Such equivalents are intended to be encompassed by the following claims.

1. A method for treating a disorder affecting ocular tissue in a subject, the method comprising administering to the ocular area of the subject a therapeutically effective amount of a nuclease system comprising a genome targeted nuclease and a guide DNA comprising at least one targeted genomic sequence.

2. The method of claim 1, wherein the nuclease is provided as a protein, RNA, DNA, or an expression vector comprising a nucleic acid that encodes the nuclease.

3. The method of claim 1, wherein the guide DNA is provided as an RNA molecule (gRNA), DNA molecule, or an expression vector comprising a nucleic acid that encodes the gRNA.

4. The method of claim 1, wherein the nuclease system is CRISPR-Cas9.

5. The method of claim 1, wherein the nuclease system inactivates or excises gene mutations.

6. The method of claim 1, further comprising a DNA double-stranded break (DSB) repair system.

7. The method of claim 6, wherein the DSB repair system comprises a repair template in combination with or without a Non-Homologous End-Joining (NHEJ) or Homology Directed Repair (HDR) targeted to the one or more CRISPR-Cas9 cleavage site, said site corrects or edits a genomic mutation.

8. The method of claim 1, wherein the genome targeted nuclease is Cas9.

9. The method of claim 1, wherein the disorder is a corneal dystrophy or microsatellite expansion disease.

10. The method of claim 1, wherein the ocular area is the cornea.

11. The method of claim 1, wherein the guide DNA comprises at least one, two, three, four, five, six, seven, eight, nine, or ten targeted genomic sequences.

12. The method of claim 11, wherein the target genomic sequences are selected from any one of the nucleotide sequences set forth in SEQ ID NOs: 1-172 and 174-342, or any combination thereof.

13. The method of claim 9, wherein the corneal dystrophy is selected from the group consisting of Epithelial Basement Membrane Dystrophy, Epithelial Recurrent Erosion Dystrophies, Subepithelial Mucinous Corneal Dystrophy, Meesmann Corneal Dystrophy, Lisch Epithelial Corneal Dystrophy, Gelatinous Drop-like Corneal Dystrophy, Reis-Bucklers Corneal Dystrophy, Thiel-Behnke Corneal Dystrophy, Lattice Corneal Dystrophy, Type 1 (Classic), Lattice Corneal Dystrophy, Type 2, Lattice Corneal Dystrophy, Type III, Lattice Corneal Dystrophy, Type IIIA, Lattice Corneal Dystrophy, Type I/IIIA, Lattice Corneal Dystrophy, Type IV, Polymorphic (Corneal) Amyloidosis, Granular Corneal Dystrophy, Type 1, Granular Corneal Dystrophy, Type 2, Macular Corneal Dystrophy, Schnyder Corneal Dystrophy, Congenital Stromal Corneal Dystrophy, Fleck Corneal Dystrophy, Posterior Amorphous Corneal Dystrophy, Central Cloudy Dystrophy of Francois, Pre-Descemet Corneal Dystrophy, Fuchs Endothelial Corneal Dystrophy, Posterior Polymorphous Corneal Dystrophy, Congenital Hereditary Endothelial Dystrophy, and X-linked Endothelial Corneal Dystrophy.

14. The method of claim 9, wherein the microsatellite expansion diseases is selected from the group consisting of Blepharophimosis, ptosis and epicanthus inversus syndactyly, Cleidocranial dysplasia, Congenital central hypoventilation syndrome, Haddad syndrome DM (Myotonic dystrophy), FRAXA (Fragile X syndrome), FRAXE (Fragile XE mental retardation), FRDA (Friedreich's ataxia), Fuchs' Endothelial Corneal Dystrophy, FXTAS (Fragile X-associated tremor/ataxia syndrome), Hand-foot-genital syndrome, HD (Huntington's disease), Holoprosencephaly, Mental retardation with growth hormone deficiency, Mental retardation, epilepsy, West syndrome, Partington syndrome, Oculopharyngeal muscular dystrophy, SBMA (Spinal and bulbar muscular atrophy), SCA1 (Spinocerebellar ataxia Type 1), SCA12 (Spinocerebellar ataxia Type 12), SCA17 (Spinocerebellar ataxia Type 17), SCA2 (Spinocerebellar ataxia Type 2), SCA3 (Spinocerebellar ataxia Type 3 or Machado-Joseph disease), SCA6 (Spinocerebellar ataxia Type 6), SCAT (Spinocerebellar ataxia Type 7), SCA8 (Spinocerebellar ataxia Type 8), and Synpolydactyly.

15. The method of claim 1, wherein the nuclease system is administered topically to the surface of the eye.

16. The method of claim 1, wherein the nuclease system is administered on or outside the cornea, sclera, to the intraocular, subconjunctival, sub-tenon, or retrobulbar space, or in or around the eyelids.

17. The method of claim 1, wherein the nuclease system is administered by implantation, injection, or virally.

18. A method for treating a disorder affecting non-ocular tissue in a subject, the method comprising administering to the non-ocular tissue of the subject a therapeutically effective amount of a nuclease system comprising a genome targeted nuclease and a guide DNA comprising at least one targeted genomic sequence.

19. The method of claim 18, wherein the nuclease is provided as a protein, RNA, DNA, or an expression vector comprising a nucleic acid encoding the nuclease.

20. The method of claim 18, wherein the guide DNA is provided as an RNA molecule (gRNA), DNA molecule, or an expression vector comprising a nucleic acid that encodes the gRNA.

21. The method of claim 18, wherein the nuclease system is CRISPR-Cas9.

22. The method of claim 18, wherein the nuclease system inactivates or excises gene mutations.

23. The method of claim 18, further comprising a DNA double-stranded break (DSB) repair system.

24. The method of claim 23, wherein the DSB repair system comprises a repair template in combination with a Non-Homologous End-Joining (NHEJ) or Homology Directed Repair (HDR) targeted to the one or more CRISPR-Cas9 cleavage site, said site corrects or edits a genomic mutation.

25. The method of claim 18, wherein the genome targeted nuclease is Cas9.

26. The method of claim 18, wherein the disorder is microsatellite expansion disease.

27. The method of claim 18, wherein the guide DNA comprises at least one, two, three, four, five, six, seven, eight, nine, or ten targeted genomic sequences.

28. The method of claim 27, wherein the target genomic sequences are selected from any one of the nucleotide sequences set forth in SEQ ID NOs: 1-172 and 174-342, or any combination thereof.

29. The method of claim 26, wherein the microsatellite expansion diseases is selected from the group consisting of Blepharophimosis, ptosis and epicanthus inversus syndactyly, Cleidocranial dysplasia, Congenital central hypoventilation syndrome, Haddad syndrome DM (Myotonic dystrophy), FRAXA (Fragile X syndrome), FRAXE (Fragile XE mental retardation), FRDA (Friedreich's ataxia), Fuchs' Endothelial Corneal Dystrophy, FXTAS (Fragile X-associated tremor/ataxia syndrome), Hand-foot-genital syndrome, HD (Huntington's disease), Holoprosencephaly, Mental retardation with growth hormone deficiency, Mental retardation, epilepsy, West syndrome, Partington syndrome, Oculopharyngeal muscular dystrophy, SBMA (Spinal and bulbar muscular atrophy), SCA1 (Spinocerebellar ataxia Type 1), SCA12 (Spinocerebellar ataxia Type 12), SCA17 (Spinocerebellar ataxia Type 17), SCA2 (Spinocerebellar ataxia Type 2), SCA3 (Spinocerebellar ataxia Type 3 or Machado-Joseph disease), SCA6 (Spinocerebellar ataxia Type 6), SCAT (Spinocerebellar ataxia Type 7), SCA8 (Spinocerebellar ataxia Type 8), and Synpolydactyly.

30. The method of claim 18, wherein the nuclease system is administered topically, intravascularly, intradermally, transdermally, parenterally, intravenously, intramuscularly, intranasally, subcutaneously, regionally, percutaneously, intratracheally, intraperitoneally, intraarterially, intravesically, intratumorally, inhalationly, perfusionly, lavagely, directly via injection, or orally via administration and formulation.


SEQUENCE LISTING (374 sequences)

SEQ ID NO: 1 - DNA, 23 bp, Homo sapiens
gagtgatgacaggacacttgtgg

SEQ ID NO: 2 - DNA, 23 bp, Homo sapiens
tggattcaagtacgtggctctgg

SEQ ID NO: 3 - DNA, 23 bp, Homo sapiens
tcctgtcatcactctttttgtgg

SEQ ID NO: 4 - DNA, 23 bp, Homo sapiens
tctggctcctttctctacacagg

SEQ ID NO: 5 - DNA, 23 bp, Homo sapiens
ggctcctttctctacacaggagg

SEQ ID NO: 6 - DNA, 23 bp, Homo sapiens
ggctcctttctctacacaggagg

SEQ ID NO: 7 - DNA, 23 bp, Homo sapiens
gttggaatggagaatggcctcgg

SEQ ID NO: 8 - DNA, 23 bp, Homo sapiens
tctacacaggaggtaagatttgg

SEQ ID NO: 9 - DNA, 23 bp, Homo sapiens
tctacacaggaggtaagatttgg

SEQ ID NO: 10 - DNA, 23 bp, Homo sapiens
ggctcctttctctacacaggagg

SEQ ID NO: 11 - DNA, 23 bp, Homo sapiens
cttacctcctgtgtagagaaagg

SEQ ID NO: 12 - DNA, 23 bp, Homo sapiens
tctctggattttctggccgcagg

SEQ ID NO: 13 - DNA, 23 bp, Homo sapiens
gattttctggccgcaggaattgg

SEQ ID NO: 14 - DNA, 23 bp, Homo sapiens
aggaattggattcaagtacgtgg

SEQ ID NO: 15 - DNA, 23 bp, Homo sapiens
gtaagtgttgaccaaattaccgg

SEQ ID NO: 16 - DNA, 23 bp, Homo sapiens
acccgaataagaagcctttttgg

SEQ ID NO: 17 - DNA, 23 bp, Homo sapiens
aggaattggattcaagtacgtgg

SEQ ID NO: 18 - DNA, 23 bp, Homo sapiens
tctccattccaacaacaccaggg

SEQ ID NO: 19 - DNA, 23 bp, Homo sapiens
ggattcaagtacgtggctctggg

SEQ ID NO: 20 - DNA, 23 bp, Homo sapiens
tgtagagaaaggagccagacagg

SEQ ID NO: 21 - DNA, 23 bp, Homo sapiens
gtacttgaatccaattcctgcgg

SEQ ID NO: 22 - DNA, 23 bp, Homo sapiens
ttctccattccaacaacaccagg

SEQ ID NO: 23 - DNA, 23 bp, Homo sapiens
tctccattccaacaacaccaggg

SEQ ID NO: 24 - DNA, 23 bp, Homo sapiens
ttccaacaacaccagggacatgg

SEQ ID NO: 25 - DNA, 23 bp, Homo sapiens
ccagggacatggagtccgaccgg

SEQ ID NO: 26 - DNA, 23 bp, Homo sapiens
ggtgttgttggaatggagaatgg

SEQ ID NO: 27 - DNA, 23 bp, Homo sapiens
cagggacatggagtccgaccggg

SEQ ID NO: 28 - DNA, 23 bp, Homo sapiens
ggacatggagtccgaccgggagg

SEQ ID NO: 29 - DNA, 23 bp, Homo sapiens
ctttttgtggtcaatgcccttgg

SEQ ID NO: 30 - DNA, 23 bp, Homo sapiens
accacaaaaagagtgatgacagg

SEQ ID NO: 31 - DNA, 23 bp, Homo sapiens
tgtccctggtgttgttggaatgg

SEQ ID NO: 32 - DNA, 23 bp, Homo sapiens
ctccatgtccctggtgttgttgg

SEQ ID NO: 33 - DNA, 23 bp, Homo sapiens
ctccatgtccctggtgttgttgg

SEQ ID NO: 34 - DNA, 23 bp, Homo sapiens
tgtagagaaaggagccagacagg

SEQ ID NO: 35 - DNA, 23 bp, Homo sapiens
ccggtcggactccatgtccctgg

SEQ ID NO: 36 - DNA, 23 bp, Homo sapiens
ccaagtcacctttctgccccagg

SEQ ID NO: 37 - DNA, 23 bp, Homo sapiens
caagtcacctttctgccccaggg

SEQ ID NO: 38 - DNA, 23 bp, Homo sapiens
caagtcacctttctgccccaggg

SEQ ID NO: 39 - DNA, 23 bp, Homo sapiens
gcaaacgattctgattccccagg

SEQ ID NO: 40 - DNA, 23 bp, Homo sapiens
aaggtgacttggggctccctggg

SEQ ID NO: 41 - DNA, 23 bp, Homo sapiens
aaaggtgacttggggctccctgg

SEQ ID NO: 42 - DNA, 23 bp, Homo sapiens
cccagggctcctgccacccctgg

SEQ ID NO: 43 - DNA, 23 bp, Homo sapiens
tggggcagaaaggtgacttgggg

SEQ ID NO: 44 - DNA, 23 bp, Homo sapiens
ctggggcagaaaggtgacttggg

SEQ ID NO: 45 - DNA, 23 bp, Homo sapiens
cctggggcagaaaggtgacttgg

SEQ ID NO: 46 - DNA, 23 bp, Homo sapiens
gcaggagccctggggcagaaagg

SEQ ID NO: 47 - DNA, 23 bp, Homo sapiens
caggggtggcaggagccctgggg

SEQ ID NO: 48 - DNA, 23 bp, Homo sapiens
ccaggggtggcaggagccctggg

SEQ ID NO: 49 - DNA, 23 bp, Homo sapiens
ttggggctccctgggcagcctgg

SEQ ID NO: 50 - DNA, 23 bp, Homo sapiens
agggaatgctaagaactgctggg

SEQ ID NO: 51 - DNA, 23 bp, Homo sapiens
gaatgctaagaactgctgggagg

SEQ ID NO: 52 - DNA, 23 bp, Homo sapiens
aacgattctgattccccaggtgg

SEQ ID NO: 53 - DNA, 23 bp, Homo sapiens
gagtaggtgtatgccacctgggg

SEQ ID NO: 54 - DNA, 23 bp, Homo sapiens
tgagtaggtgtatgccacctggg

SEQ ID NO: 55 - DNA, 23 bp, Homo sapiens
aagggaatgctaagaactgctgg

SEQ ID NO: 56 - DNA, 23 bp, Homo sapiens
ctctccatgctgctcggcctcgg

SEQ ID NO: 57 - DNA, 23 bp, Homo sapiens
atgctgctcggcctcggcaatgg

SEQ ID NO: 58 - DNA, 23 bp, Homo sapiens
ggccgagcagcatggagagatgg

SEQ ID NO: 59 - DNA, 23 bp, Homo sapiens
attgccgaggccgagcagcatgg

SEQ ID NO: 60 - DNA, 23 bp, Homo sapiens
ctccgccagctcccccagagtgg

SEQ ID NO: 61 - DNA, 23 bp, Homo sapiens
tccgccagctcccccagagtggg

SEQ ID NO: 62 - DNA, 23 bp, Homo sapiens
ggagctggcggaggggcctatgg

SEQ ID NO: 63 - DNA, 23 bp, Homo sapiens
ctctgggggagctggcggagggg

SEQ ID NO: 64 - DNA, 23 bp, Homo sapiens
actctgggggagctggcggaggg

SEQ ID NO: 65 - DNA, 23 bp, Homo sapiens
cactctgggggagctggcggagg

SEQ ID NO: 66 - DNA, 23 bp, Homo sapiens
gcccactctgggggagctggcgg

SEQ ID NO: 67 - DNA, 23 bp, Homo sapiens
gtggcccactctgggggagctgg

SEQ ID NO: 68 - DNA, 23 bp, Homo sapiens
agctgtgtggcccactctggggg

SEQ ID NO: 69 - DNA, 23 bp, Homo sapiens
ggccatctctccatgctgctcgg

SEQ ID NO: 70 - DNA, 23 bp, Homo sapiens
gcctttttggtgttgtgtccagg

SEQ ID NO: 71 - DNA, 23 bp, Homo sapiens
tttttggtgttgtgtccaggtgg

SEQ ID NO: 72 - DNA, 23 bp, Homo sapiens
tttttggtgttgtgtccaggtgg

SEQ ID NO: 73 - DNA, 23 bp, Homo sapiens
gcctttttggtgttgtgtccagg

SEQ ID NO: 74 - DNA, 23 bp, Homo sapiens
ttttggtgttgtgtccaggtggg

SEQ ID NO: 75 - DNA, 23 bp, Homo sapiens
accaaaaaggcttcttattcggg

SEQ ID NO: 76 - DNA, 23 bp, Homo sapiens
caccaaaaaggcttcttattcgg

SEQ ID NO: 77 - DNA, 23 bp, Homo sapiens
caccaaaaaggcttcttattcgg

SEQ ID NO: 78 - DNA, 23 bp, Homo sapiens
tttttggtgttgtgtccaggtgg

SEQ ID NO: 79 - DNA, 23 bp, Homo sapiens
acctggacacaacaccaaaaagg

SEQ ID NO: 80 - DNA, 23 bp, Homo sapiens
tcaagtgacttctgcccacctgg

SEQ ID NO: 81 - DNA, 23 bp, Homo sapiens
acccgaataagaagcctttttgg

SEQ ID NO: 82 - DNA, 23 bp, Homo sapiens
tcaagtgacttctgcccacctgg

SEQ ID NO: 83 - DNA, 23 bp, Homo sapiens
gagttcactgagtcactgtcggg

SEQ ID NO: 84 - DNA, 23 bp, Homo sapiens
cttgggcctccccgtccagcagg

SEQ ID NO: 85 - DNA, 23 bp, Homo sapiens
gggcctccccgtccagcaggcgg

SEQ ID NO: 86 - DNA, 23 bp, Homo sapiens
cctccccgtccagcaggcggcgg

SEQ ID NO: 87 - DNA, 23 bp, Homo sapiens
cccgtccagcaggcggcggtagg

SEQ ID NO: 88 - DNA, 23 bp, Homo sapiens
ctgctggacggggaggcccaagg

SEQ ID NO: 89 - DNA, 23 bp, Homo sapiens
ccgccgcctgctggacggggagg

SEQ ID NO: 90 - DNA, 23 bp, Homo sapiens
ctaccgccgcctgctggacgggg

SEQ ID NO: 91 - DNA, 23 bp, Homo sapiens
cctaccgccgcctgctggacggg

SEQ ID NO: 92 - DNA, 23 bp, Homo sapiens
acctaccgccgcctgctggacgg

SEQ ID NO: 93 - DNA, 23 bp, Homo sapiens
tctcaatctccagctccaggcgg

SEQ ID NO: 94 - DNA, 23 bp, Homo sapiens
ctcaatctccagctccaggcggg

SEQ ID NO: 95 - DNA, 23 bp, Homo sapiens
aggtctcaatctccagctccagg

SEQ ID NO: 96 - DNA, 23 bp, Homo sapiens
ctcaatctccagctccaggcggg

SEQ ID NO: 97 - DNA, 23 bp, Homo sapiens
ctagagctcgcaccttatccagg

SEQ ID NO: 98 - DNA, 23 bp, Homo sapiens
tgagacctaccgccgcctgctgg

SEQ ID NO: 99 - DNA, 23 bp, Homo sapiens
cctccccgtccagcaggcggcgg

SEQ ID NO: 100 - DNA, 23 bp, Homo sapiens
tgagacctaccgccgcctgctgg

SEQ ID NO: 101 - DNA, 23 bp, Homo sapiens
aggtctcaatctccagctccagg

SEQ ID NO: 102 - DNA, 23 bp, Homo sapiens
tgagacctaccgccgcctgctgg

SEQ ID NO: 103 - DNA, 23 bp, Homo sapiens
ctaccgccgcctgctggacgggg

SEQ ID NO: 104 - DNA, 23 bp, Homo sapiens
tgagacctaccgccgcctgctgg

SEQ ID NO: 105 - DNA, 23 bp, Homo sapiens
tgatagattagcttcctacctgg

SEQ ID NO: 106 - DNA, 23 bp, Homo sapiens
tgatagattagcttcctacctgg

SEQ ID NO: 107 - DNA, 23 bp, Homo sapiens
taaggtgcgagctctagaagagg

SEQ ID NO: 108 - DNA, 23 bp, Homo sapiens
ctagagctcgcaccttatccagg

SEQ ID NO: 109 - DNA, 23 bp, Homo sapiens
agctcgcaccttatccaggtagg

SEQ ID NO: 110 - DNA, 23 bp, Homo sapiens
agctcgcaccttatccaggtagg

SEQ ID NO: 111 - DNA, 23 bp, Homo sapiens
attagcttcctacctggataagg

SEQ ID NO: 112 - DNA, 23 bp, Homo sapiens
attagcttcctacctggataagg

SEQ ID NO: 113 - DNA, 23 bp, Homo sapiens
tgatagattagcttcctacctgg

SEQ ID NO: 114 - DNA, 23 bp, Homo sapiens
ctaccgccgcctgctggacgggg

SEQ ID NO: 115 - DNA, 23 bp, Homo sapiens
tgatagattagcttcctacctgg

SEQ ID NO: 116 - DNA, 23 bp, Homo sapiens
tgatagattagcttcctacctgg

SEQ ID NO: 117 - DNA, 23 bp, Homo sapiens
tgctggacggggaggcccaaggg

SEQ ID NO: 118 - DNA, 23 bp, Homo sapiens
cactctgcgaggagagccgccgg

SEQ ID NO: 119 - DNA, 23 bp, Homo sapiens
actctgcgaggagagccgccggg

SEQ ID NO: 120 - DNA, 23 bp, Homo sapiens
ggagagccgccgggacagtccgg

SEQ ID NO: 121 - DNA, 23 bp, Homo sapiens
gagagccgccgggacagtccggg

SEQ ID NO: 122 - DNA, 23 bp, Homo sapiens
agagccgccgggacagtccgggg

SEQ ID NO: 123 - DNA, 23 bp, Homo sapiens
gagccgccgggacagtccggggg

SEQ ID NO: 124 - DNA, 23 bp, Homo sapiens
gcacccccggactgtcccggcgg

SEQ ID NO: 125 - DNA, 23 bp, Homo sapiens
tgcgcacccccggactgtcccgg

SEQ ID NO: 126 - DNA, 23 bp, Homo sapiens
tgcgcacccccggactgtcccgg

SEQ ID NO: 127 - DNA, 23 bp, Homo sapiens
ggagagccgccgggacagtccgg

SEQ ID NO: 128 - DNA, 23 bp, Homo sapiens
tcactctcagtgcgcacccccgg

SEQ ID NO: 129 - DNA, 23 bp, Homo sapiens
taaggtgcgagctctagaagagg

SEQ ID NO: 130 - DNA, 23 bp, Homo sapiens
cagtgactcagtgaactccgtgg

SEQ ID NO: 131 - DNA, 23 bp, Homo sapiens
gactcagtgaactccgtggaagg

SEQ ID NO: 132 - DNA, 23 bp, Homo sapiens
gcaaaagcaatgatggagctggg

SEQ ID NO: 133 - DNA, 23 bp, Homo sapiens
ggagttcactgagtcactgtcgg

SEQ ID NO: 134 - DNA, 23 bp, Homo sapiens
gatgaccaacaagatgctttagg

SEQ ID NO: 135 - DNA, 23 bp, Homo sapiens
catcttgttggtcatccacaggg

SEQ ID NO: 136 - DNA, 23 bp, Homo sapiens
gcatcttgttggtcatccacagg

SEQ ID NO: 137 - DNA, 23 bp, Homo sapiens
cgctgcctaaagcatcttgttgg

SEQ ID NO: 138 - DNA, 23 bp, Homo sapiens
ggaggaagaaacagctgctcagg

SEQ ID NO: 139 - DNA, 23 bp, Homo sapiens
gaggaagaaacagctgctcaggg

SEQ ID NO: 140 - DNA, 23 bp, Homo sapiens
atggaagagaaactcatgatcgg

SEQ ID NO: 141 - DNA, 23 bp, Homo sapiens
ccatcattgcttttgctctcagg

SEQ ID NO: 142 - DNA, 23 bp, Homo sapiens
agcaaaagcaatgatggagctgg

SEQ ID NO: 143 - DNA, 23 bp, Homo sapiens
cctgagagcaaaagcaatgatgg

SEQ ID NO: 144 - DNA, 23 bp, Homo sapiens
tgttggtcatccacagggagtgg

SEQ ID NO: 145 - DNA, 23 bp, Homo sapiens
tcagctgtacacggaccgcacgg

SEQ ID NO: 146 - DNA, 23 bp, Homo sapiens
cggaccgcacggagaagctgagg

SEQ ID NO: 147 - DNA, 23 bp, Homo sapiens
caggcctcagcttctccgtgcgg

SEQ ID NO: 148 - DNA, 23 bp, Homo sapiens
gcggtccgtgtacagctgagtgg

SEQ ID NO: 149 - DNA, 23 bp, Homo sapiens
caggcctcagcttctccgtgcgg

SEQ ID NO: 150 - DNA, 23 bp, Homo sapiens
gttctcttggtggcagggctcgg

SEQ ID NO: 151 - DNA, 23 bp, Homo sapiens
caggcctcagcttctccgtgcgg

SEQ ID NO: 152 - DNA, 23 bp, Homo sapiens
cctgacatcatggccacaaatgg

SEQ ID NO: 153 - DNA, 23 bp, Homo sapiens
cgggtgctgacccccccaatggg

SEQ ID NO: 154 - DNA, 23 bp, Homo sapiens
gggtgctgacccccccaatgggg

SEQ ID NO: 155 - DNA, 23 bp, Homo sapiens
ccccccaatggggactgtcatgg

SEQ ID NO: 156 - DNA, 23 bp, Homo sapiens
ccattgggggggtcagcacccgg

SEQ ID NO: 157 - DNA, 23 bp, Homo sapiens
actgtcatggatgtcctgaaggg

SEQ ID NO: 158 - DNA, 23 bp, Homo sapiens
catgacagtccccattggggggg

SEQ ID NO: 159 - DNA, 23 bp, Homo sapiens
ccatgacagtccccattgggggg

SEQ ID NO: 160 - DNA, 23 bp, Homo sapiens
tccatgacagtccccattggggg

SEQ ID NO: 161 - DNA, 23 bp, Homo sapiens
atccatgacagtccccattgggg

SEQ ID NO: 162 - DNA, 23 bp, Homo sapiens
catccatgacagtccccattggg

SEQ ID NO: 163 - DNA, 23 bp, Homo sapiens
acatccatgacagtccccattgg

SEQ ID NO: 164 - DNA, 23 bp, Homo sapiens
taaagcgattgtctcccttcagg

SEQ ID NO: 165 - DNA, 23 bp, Homo sapiens
agactgtgtagactccttcccgg

SEQ ID NO: 166 - DNA, 23 bp, Homo sapiens
gcggtccgtgtacagctgagtgg

SEQ ID NO: 167 - DNA, 23 bp, Homo sapiens
gagccctgccaccaagagaacgg

SEQ ID NO: 168 - DNA, 23 bp, Homo sapiens
ggctcggaaggcttcatttgtgg

SEQ ID NO: 169 - DNA, 23 bp, Homo sapiens
gcggtccgtgtacagctgagtgg

SEQ ID NO: 170 - DNA, 23 bp, Homo sapiens
aagagaacggagcagactcttgg

SEQ ID NO: 171 - DNA, 23 bp, Homo sapiens
agagaacggagcagactcttggg

SEQ ID NO: 172 - DNA, 23 bp, Homo sapiens
ccgggtgctgacccccccaatgg

SEQ ID NO: 173 - DNA, 53 bp, Artificial Sequence
  Description of Artificial Sequence Synthetic primer
cttataagttctgtatgagaccactttttccctcagctgtacacggaccgcag

SEQ ID NO: 174 - DNA, 23 bp, Homo sapiens
gctcggaaggcttcatttgtggg

SEQ ID NO: 175 - DNA, 23 bp, Homo sapiens
gctccgttctcttggtggcaggg

SEQ ID NO: 176 - DNA, 23 bp, Homo sapiens
tgctccgttctcttggtggcagg

SEQ ID NO: 177 - DNA, 23 bp, Homo sapiens
agtctgctccgttctcttggtgg

SEQ ID NO: 178 - DNA, 23 bp, Homo sapiens
aagagtctgctccgttctcttgg

SEQ ID NO: 179 - DNA, 23 bp, Homo sapiens
catcatggccacaaatggcgtgg

SEQ ID NO: 180 - DNA, 23 bp, Homo sapiens
ccatttgtggccatgatgtcagg

SEQ ID NO: 181 - DNA, 23 bp, Homo sapiens
gacatggaccacgccatttgtgg

SEQ ID NO: 182 - DNA, 23 bp, Homo sapiens
gcggtccgtgtacagctgagtgg

SEQ ID NO: 183 - DNA, 23 bp, Homo sapiens
ttttctttcaggcttcccagagg

SEQ ID NO: 184 - DNA, 23 bp, Homo sapiens
ttttctttcaggcttcccagagg

SEQ ID NO: 185 - DNA, 23 bp, Homo sapiens
gactgtcatggatgtcctgaagg

SEQ ID NO: 186 - DNA, 23 bp, Homo sapiens
acctagtcgcacagacctctggg

SEQ ID NO: 187 - DNA, 23 bp, Homo sapiens
aggaggaaaagcaaacaactggg

SEQ ID NO: 188 - DNA, 23 bp, Homo sapiens
ggaaaagcaaacaactgggaagg

SEQ ID NO: 189 - DNA, 23 bp, Homo sapiens
aaagcaaacaactgggaaggagg

SEQ ID NO: 190 - DNA, 23 bp, Homo sapiens
acaactgggaaggaggtatccgg

SEQ ID NO: 191 - DNA, 23 bp, Homo sapiens
caactgggaaggaggtatccggg

SEQ ID NO: 192 - DNA, 23 bp, Homo sapiens
tggggcagaaaggtgacttgggg

SEQ ID NO: 193 - DNA, 23 bp, Homo sapiens
ttaagtaggcgttgcagtaatgg

SEQ ID NO: 194 - DNA, 23 bp, Homo sapiens
taggaggaaaagcaaacaactgg

SEQ ID NO: 195 - DNA, 23 bp, Homo sapiens
aaagagccccacttggaaggcgg

SEQ ID NO: 196 - DNA, 23 bp, Homo sapiens
gccccacttggaaggcggtttgg

SEQ ID NO: 197 - DNA, 23 bp, Homo sapiens
gattttatttgtgtgttttgtgg

SEQ ID NO: 198 - DNA, 23 bp, Homo sapiens
tccaaaccgccttccaagtgggg

SEQ ID NO: 199 - DNA, 23 bp, Homo sapiens
atccaaaccgccttccaagtggg

SEQ ID NO: 200 - DNA, 23 bp, Homo sapiens
aatccaaaccgccttccaagtgg

SEQ ID NO: 201 - DNA, 23 bp, Homo sapiens
catcttacaccaaactcatctgg

SEQ ID NO: 202 - DNA, 23 bp, Homo sapiens
tttttaatgccagatgagtttgg

SEQ ID NO: 203 - DNA, 23 bp, Homo sapiens
attcattctcctgacatgtctgg

SEQ ID NO: 204 - DNA, 23 bp, Homo sapiens
ttcattctcctgacatgtctggg

SEQ ID NO: 205 - DNA, 23 bp, Homo sapiens
ctcctgacatgtctgggacttgg

SEQ ID NO: 206 - DNA, 23 bp, Homo sapiens
acatgtctgggacttggtttagg

SEQ ID NO: 207 - DNA, 23 bp, Homo sapiens
ctgggacttggtttaggaaaagg

SEQ ID NO: 208 - DNA, 23 bp, Homo sapiens
ggtttaggaaaaggaagcaaagg

SEQ ID NO: 209 - DNA, 23 bp, Homo sapiens
gtttaggaaaaggaagcaaaggg

SEQ ID NO: 210 - DNA, 23 bp, Homo sapiens
aaccaagtcccagacatgtcagg

SEQ ID NO: 211 - DNA, 23 bp, Homo sapiens
aggaaaaggaagcaaagggatgg

SEQ ID NO: 212 - DNA, 23 bp, Homo sapiens
aggaagcaaagggatggagaagg

SEQ ID NO: 213 - DNA, 23 bp, Homo sapiens
tggagttttacggctgtacttgg

SEQ ID NO: 214 - DNA, 23 bp, Homo sapiens
gacacacttgtggagttttacgg

SEQ ID NO: 215 - DNA, 23 bp, Homo sapiens
agcggaacttgacacacttgtgg

SEQ ID NO: 216 - DNA, 23 bp, Homo sapiens
gtcgtaggatcagcacaaagcgg

SEQ ID NO: 217 - DNA, 23 bp, Homo sapiens
atttaccaaaacagtccaaaagg

SEQ ID NO: 218 - DNA, 23 bp, Homo sapiens
ttggtaaatttcgtagtcgtagg

SEQ ID NO: 219 - DNA, 23 bp, Homo sapiens
tagaaccttttggactgttttgg

SEQ ID NO: 220 - DNA, 23 bp, Homo sapiens
atacattctttagaaccttttgg

SEQ ID NO: 221 - DNA, 23 bp, Homo sapiens
atactagttttaagaatcctagg

SEQ ID NO: 222 - DNA, 23 bp, Homo sapiens
tcctaggaaaagatgtaactagg

SEQ ID NO: 223 - DNA, 23 bp, Homo sapiens
taggaaaagatgtaactaggagg

SEQ ID NO: 224 - DNA, 23 bp, Homo sapiens
taggattcttaaaactagtatgg

SEQ ID NO: 225 - DNA, 23 bp, Homo sapiens
taactaggaggtaagatgtaagg

SEQ ID NO: 226 - DNA, 23 bp, Homo sapiens
ggaggtaagatgtaaggaacagg

SEQ ID NO: 227 - DNA, 23 bp, Homo sapiens
tcctagttacatcttttcctagg

SEQ ID NO: 228 - DNA, 23 bp, Homo sapiens
taatgatgctttggattggtagg

SEQ ID NO: 229 - DNA, 23 bp, Homo sapiens
aagctaatgatgctttggattgg

SEQ ID NO: 230 - DNA, 23 bp, Homo sapiens
taaaactttaaagagacaactgg

SEQ ID NO: 231 - DNA, 23 bp, Homo sapiens
aaaactttaaagagacaactggg

SEQ ID NO: 232 - DNA, 23 bp, Homo sapiens
gttttaagctaatgatgctttgg

SEQ ID NO: 233 - DNA, 23 bp, Homo sapiens
ggaaatggaaaatagaaaatagg

SEQ ID NO: 234 - DNA, 23 bp, Homo sapiens
ttatttattgtttttggaaatgg

SEQ ID NO: 235 - DNA, 23 bp, Homo sapiens
ttcgttttatttattgtttttgg

SEQ ID NO: 236 - DNA, 23 bp, Homo sapiens
gtagtctcagtgttcagacatgg

SEQ ID NO: 237 - DNA, 23 bp, Homo sapiens
ttcagacatggccaagttttagg

SEQ ID NO: 238 - DNA, 23 bp, Homo sapiens
tcagacatggccaagttttaggg

SEQ ID NO: 239 - DNA, 23 bp, Homo sapiens
cagacatggccaagttttagggg

SEQ ID NO: 240 - DNA, 23 bp, Homo sapiens
acatggccaagttttaggggtgg

SEQ ID NO: 241 - DNA, 23 bp, Homo sapiens
tttaggggtggtttagttttagg

SEQ ID NO: 242 - DNA, 23 bp, Homo sapiens
ttaggggtggtttagttttaggg

SEQ ID NO: 243 - DNA, 23 bp, Homo sapiens
taggggtggtttagttttagggg

SEQ ID NO: 244 - DNA, 23 bp, Homo sapiens
actaaaccacccctaaaacttgg

SEQ ID NO: 245 - DNA, 23 bp, Homo sapiens
tgtctatttttgctttccactgg

SEQ ID NO: 246 - DNA, 23 bp, Homo sapiens
gtctatttttgctttccactggg

SEQ ID NO: 247 - DNA, 23 bp, Homo sapiens
tctatttttgctttccactgggg

SEQ ID NO: 248 - DNA, 23 bp, Homo sapiens
tggggtgagattccattatttgg

SEQ ID NO: 249 - DNA, 23 bp, Homo sapiens
ggggtgagattccattatttggg

SEQ ID NO: 250 - DNA, 23 bp, Homo sapiens
gggtgagattccattatttgggg

SEQ ID NO: 251 - DNA, 23 bp, Homo sapiens
ccattatttggggtaatcagtgg

SEQ ID NO: 252 - DNA, 23 bp, Homo sapiens
cattatttggggtaatcagtggg

SEQ ID NO: 253 - DNA, 23 bp, Homo sapiens
atttggggtaatcagtgggtagg

SEQ ID NO: 254 - DNA, 23 bp, Homo sapiens
ataatggaatctcaccccagtgg

SEQ ID NO: 255 - DNA, 23 bp, Homo sapiens
tttggggtaatcagtgggtaggg

SEQ ID NO: 256 - DNA, 23 bp, Homo sapiens
atcagtgggtagggaattgaagg

SEQ ID NO: 257 - DNA, 23 bp, Homo sapiens
ccactgattaccccaaataatgg

SEQ ID NO: 258 - DNA, 23 bp, Homo sapiens
ttttttttgagttttattactgg

SEQ ID NO: 259 - DNA, 23 bp, Homo sapiens
tgtggtgtgatggaagattcagg

SEQ ID NO: 260 - DNA, 23 bp, Homo sapiens
actataattttgtggtgtgatgg

SEQ ID NO: 261 - DNA, 23 bp, Homo sapiens
agtttttaactataattttgtgg

SEQ ID NO: 262 - DNA, 23 bp, Homo sapiens
aaagaccttcatatttaccaagg

SEQ ID NO: 263 - DNA, 23 bp, Homo sapiens
tgaatccttggtaaatatgaagg

SEQ ID NO: 264 - DNA, 23 bp, Homo sapiens
tttttaattggctgaatccttgg

SEQ ID NO: 265 - DNA, 23 bp, Homo sapiens
actgtcctttagattcctactgg

SEQ ID NO: 266 - DNA, 23 bp, Homo sapiens
ggacagtaataatttttaattgg

SEQ ID NO: 267 - DNA, 23 bp, Homo sapiens
tggtttctagctgaagtgtttgg

SEQ ID NO: 268 - DNA, 23 bp, Homo sapiens
ggtttctagctgaagtgtttggg

SEQ ID NO: 269 - DNA, 23 bp, Homo sapiens
agaaaccagtaggaatctaaagg

SEQ ID NO: 270 - DNA, 23 bp, Homo sapiens
cacttcagctagaaaccagtagg

SEQ ID NO: 271 - DNA, 23 bp, Homo sapiens
agtgcggtaagaaagaacggtgg

SEQ ID NO: 272 - DNA, 23 bp, Homo sapiens
ttcagtgcggtaagaaagaacgg

SEQ ID NO: 273 - DNA, 23 bp, Homo sapiens
tgatttactggatttcagtgcgg

SEQ ID NO: 274 - DNA, 23 bp, Homo sapiens
agatctttgaggagctctgaagg

SEQ ID NO: 275 - DNA, 23 bp, Homo sapiens
aacagtatgaaagatctttgagg

SEQ ID NO: 276 - DNA, 23 bp, Homo sapiens
acagcttagagtttatgctaagg

SEQ ID NO: 277 - DNA, 23 bp, Homo sapiens
cagcttagagtttatgctaaggg

SEQ ID NO: 278 - DNA, 23 bp, Homo sapiens
agcataaactctaagctgtttgg

SEQ ID NO: 279 - DNA, 23 bp, Homo sapiens
tcttttagttttaagttggatgg

SEQ ID NO: 280 - DNA, 23 bp, Homo sapiens
tttctcttttagttttaagttgg

SEQ ID NO: 281 - DNA, 23 bp, Homo sapiens
gtgataatgggggctggggtggg

SEQ ID NO: 282 - DNA, 23 bp, Homo sapiens
agtgataatgggggctggggtgg

SEQ ID NO: 283 - DNA, 23 bp, Homo sapiens
tctgttctttctttttcctcagg

SEQ ID NO: 284 - DNA, 23 bp, Homo sapiens
cagagtgataatgggggctgggg

SEQ ID NO: 285 - DNA, 23 bp, Homo sapiens
acagagtgataatgggggctggg

SEQ ID NO: 286 - DNA, 23 bp, Homo sapiens
aacagagtgataatgggggctgg

SEQ ID NO: 287 - DNA, 23 bp, Homo sapiens
aaagaacagagtgataatggggg

SEQ ID NO: 288 - DNA, 23 bp, Homo sapiens
gaaagaacagagtgataatgggg

SEQ ID NO: 289 - DNA, 23 bp, Homo sapiens
agaaagaacagagtgataatggg

SEQ ID NO: 290 - DNA, 23 bp, Homo sapiens
aagaaagaacagagtgataatgg

SEQ ID NO: 291 - DNA, 23 bp, Homo sapiens
ttttcctcaggttcattagatgg

SEQ ID NO: 292 - DNA, 23 bp, Homo sapiens
ttggccatctaatgaacctgagg

SEQ ID NO: 293 - DNA, 23 bp, Homo sapiens
agcagtactactgctacatttgg

SEQ ID NO: 294 - DNA, 23 bp, Homo sapiens
aatgtagcagtagtactgcttgg

SEQ ID NO: 295 - DNA, 23 bp, Homo sapiens
ccataatgttatcaagattcagg

SEQ ID NO: 296 - DNA, 23 bp, Homo sapiens
aatgttatcaagattcaggttgg

SEQ ID NO: 297 - DNA, 23 bp, Homo sapiens
gttatcaagattcaggttggagg

SEQ ID NO: 298 - DNA, 23 bp, Homo sapiens
tgaatcttgataacattatgggg

SEQ ID NO: 299 - DNA, 23 bp, Homo sapiens
ctgaatcttgataacattatggg

SEQ ID NO: 300 - DNA, 23 bp, Homo sapiens
cctgaatcttgataacattatgg

SEQ ID NO: 301 - DNA, 23 bp, Homo sapiens
gaaaaacactagtttcaccaagg

SEQ ID NO: 302 - DNA, 23 bp, Homo sapiens
tgtttttctagagaggctgctgg

SEQ ID NO: 303 - DNA, 23 bp, Homo sapiens
aaactagtgtttttctagagagg

SEQ ID NO: 304 - DNA, 23 bp, Homo sapiens
aacaacttttttcttctccttgg

SEQ ID NO: 305 - DNA, 23 bp, Homo sapiens
ttgttttatattgaaaaccttgg

SEQ ID NO: 306 - DNA, 23 bp, Homo sapiens
gaaaaccttggccataaacgtgg

SEQ ID NO: 307 - DNA, 23 bp, Homo sapiens
tgtccatttccatctcgtatagg

SEQ ID NO: 308 - DNA, 23 bp, Homo sapiens
cattgccacgtttatggccaagg

SEQ ID NO: 309 - DNA, 23 bp, Homo sapiens
aatggacattgccacgtttatgg

SEQ ID NO: 310 - DNA, 23 bp, Homo sapiens
aatcctatacgagatggaaatgg

SEQ ID NO: 311 - DNA, 23 bp, Homo sapiens
caggcaaatcctatacgagatgg

SEQ ID NO: 312 - DNA, 23 bp, Homo sapiens
tatttgggttcacatatgacagg

SEQ ID NO: 313 - DNA, 23 bp, Homo sapiens
tggcacttttatttttatttggg

SEQ ID NO: 314 - DNA, 23 bp, Homo sapiens
gtggcacttttatttttatttgg

SEQ ID NO: 315 - DNA, 23 bp, Homo sapiens
attctcatttcgtctctaacagg

SEQ ID NO: 316 - DNA, 23 bp, Homo sapiens
aaatgagaatttagtgcaggtgg

SEQ ID NO: 317 - DNA, 23 bp, Homo sapiens
acgaaatgagaatttagtgcagg

SEQ ID NO: 318 - DNA, 23 bp, Homo sapiens
gcatttatttcgaccctaattgg

SEQ ID NO: 319 - DNA, 23 bp, Homo sapiens
ctcttcttcgacgtatctagtgg

SEQ ID NO: 320 - DNA, 23 bp, Homo sapiens
aagaagagggaaaccaattaggg

SEQ ID NO: 321 - DNA, 23 bp, Homo sapiens
gaagaagagggaaaccaattagg

SEQ ID NO: 322 - DNA, 23 bp, Homo sapiens
actagatacgtcgaagaagaggg

SEQ ID NO: 323 - DNA, 23 bp, Homo sapiens
cactagatacgtcgaagaagagg

SEQ ID NO: 324 - DNA, 23 bp, Homo sapiens
tcagagcctgcaaaaagcaaagg

SEQ ID NO: 325 - DNA, 23 bp, Homo sapiens
gcaaaaagcaaaggaacgaatgg

SEQ ID NO: 326 - DNA, 23 bp, Homo sapiens
tgcaggctctgactcagggaagg

SEQ ID NO: 327 - DNA, 23 bp, Homo sapiens
tttttgcaggctctgactcaggg

SEQ ID NO: 328 - DNA, 23 bp, Homo sapiens
ctttttgcaggctctgactcagg

SEQ ID NO: 329 - DNA, 23 bp, Homo sapiens
ttcgttcctttgctttttgcagg

SEQ ID NO: 330 - DNA, 23 bp, Homo sapiens
agaaagtgcaacaagcagaaagg

SEQ ID NO: 331 - DNA, 23 bp, Homo sapiens
gaaagtgcaacaagcagaaaggg

SEQ ID NO: 332 - DNA, 23 bp, Homo sapiens
aaagtgcaacaagcagaaagggg

SEQ ID NO: 333 - DNA, 23 bp, Homo sapiens
aagtgcaacaagcagaaaggggg

SEQ ID NO: 334 - DNA, 23 bp, Homo sapiens
ggctgcaaagctgcctgcctagg

SEQ ID NO: 335 - DNA, 23 bp, Homo sapiens
gctgcaaagctgcctgcctaggg

SEQ ID NO: 336 - DNA, 23 bp, Homo sapiens
ctgcctagggctacgtttcctgg

SEQ ID NO: 337 - DNA, 23 bp, Homo sapiens
caggaaacgtagccctaggcagg

SEQ ID NO: 338 - DNA, 23 bp, Homo sapiens
ttgccaggaaacgtagccctagg

SEQ ID NO: 339 - DNA, 23 bp, Homo sapiens
aaagccatttctccaaaagaagg

SEQ ID NO: 340 - DNA, 23 bp, Homo sapiens
tggctttcggaagttttgccagg

SEQ ID NO: 341 - DNA, 23 bp, Homo sapiens
tcttttggagaaatggctttcgg

SEQ ID NO: 342 - DNA, 23 bp, Homo sapiens
tagaccttcttttggagaaatgg

SEQ ID NO: 343 - DNA, 56 bp, Artificial Sequence
  Description of Artificial Sequence Synthetic primer
ccttattttaacttgctatttctagctctaaaactgcggtccgtgtacagctgagg

SEQ ID NO: 344 - DNA, 53 bp, Artificial Sequence
  Description of Artificial Sequence Synthetic primer
cttataagttctgtatgagaccactttttcccagagaacggagcagactcttg

SEQ ID NO: 345 - DNA, 56 bp, Artificial Sequence
  Description of Artificial Sequence Synthetic primer
ccttattttaacttgctatttctagctctaaaacaagagtctgctccgttctctgg

SEQ ID NO: 346 - DNA, 25 bp, Artificial Sequence
  Description of Artificial Sequence Synthetic primer
ccacctgtagatgtaccgtgctctc

SEQ ID NO: 347 - DNA, 25 bp, Artificial Sequence
  Description of Artificial Sequence Synthetic primer
aggggctgcagactctgtgtttaag

SEQ ID NO: 348 - DNA, 25 bp, Artificial Sequence
  Description of Artificial Sequence Synthetic primer
aaggaaaatacctctcagcgtggtg

SEQ ID NO: 349 - DNA, 25 bp, Artificial Sequence
  Description of Artificial Sequence Synthetic primer
aggcctaggggtagtaaaggcttcc

SEQ ID NO: 350 - DNA, 588 bp, Artificial Sequence
  Description of Artificial Sequence Synthetic polynucleotide
ccacctgtagatgtaccgtgctctctgtcagagaagggagggtgtggttgggctggacccccagaggccatccctccttctgtcttctgctcctgcagccctaccactctcaaacctttacgagaccctgggagtcgttggatccaccaccactcagctgtacacggaccgcacggagaagctgaggcctgagatggaggggcccggcagcttcaccatcttcgcccctagcaacgaggcctgggcctccttgccagctgtgagatgacctccgtctgcccgggggactcttatggggaactgccttacttccccgaggggtgggcatgatgaatgggagtctgcagtcatttcctactgtttcaggaagctttctccttaaccccttagaaaaggctgtggaacttgagctaaaatatgtcttaccaggttgcgtctaatgccccccgttccctactgggcagaaagacttgggtgcttcctgaggagggatccttggcagaagagaggcctgggctcacgagggctgagaacatgtttcccagagttgcaaggacccatctcttaaacacagagtctgcagcccct

SEQ ID NO: 351 - DNA, 424 bp, Artificial Sequence
  Description of Artificial Sequence Synthetic polynucleotide
aaggaaaatacctctcagcgtggtgaggtatttaaggaaaatacctgttgacaggtgacattttctgtgtgtgtatctacagcatgctggtagctgccatccagtctgcaggactgacggagaccctcaaccgggaaggagtctacacagtctttgctcccacaaatgaagccttccgagccctgccaccaagagaacggagcagactcttgggtaaagaccaacttaagtacacgtctccatttttctaaagtagtgatccctcagggccccagcagcaaacagttggcacatcaaggattgacttgaagggattttatgacaagactattagtgaaagagtgggcgggactaaaggaactagcaaaggatgaggccaaccagggactagcaaccctgggaagcctttactacccctaggcct

SEQ ID NO: 352 - DNA, 53 bp, Artificial Sequence
  Description of Artificial Sequence Synthetic primer
cttataagttctgtatgagaccactttttcccttgccaggaaacgtagccctg

SEQ ID NO: 353 - DNA, 55 bp, Artificial Sequence
  Description of Artificial Sequence Synthetic primer
ccttattttaacttgctatttctagctctaaaacagggctacgtttcctggcaag

SEQ ID NO: 354 - DNA, 53 bp, Artificial Sequence
  Description of Artificial Sequence Synthetic primer
cttataagttctgtatgagaccactttttccccaggaaacgtagccctaggcg

SEQ ID NO: 355 - DNA, 55 bp, Artificial Sequence
  Description of Artificial Sequence Synthetic primer
ccttattttaacttgctatttctagctctaaaacgcctagggctacgtttcctgg

SEQ ID NO: 356 - DNA, 53 bp, Artificial Sequence
  Description of Artificial Sequence Synthetic primer
cttataagttctgtatgagaccactttttcccctgcctagggctacgtttccg

SEQ ID NO: 357 - DNA, 55 bp, Artificial Sequence
  Description of Artificial Sequence Synthetic primer
ccttattttaacttgctatttctagctctaaaacggaaacgtagccctaggcagg

SEQ ID NO: 358 - DNA, 53 bp, Artificial Sequence
  Description of Artificial Sequence Synthetic primer
cttataagttctgtatgagaccactttttcccgctgcaaagctgcctgcctag

SEQ ID NO: 359 - DNA, 55 bp, Artificial Sequence
  Description of Artificial Sequence Synthetic primer
ccttattttaacttgctatttctagctctaaaactaggcaggcagctttgcagcg

SEQ ID NO: 360 - DNA, 53 bp, Artificial Sequence
  Description of Artificial Sequence Synthetic primer
cttataagttctgtatgagaccactttttcccggctgcaaagctgcctgcctg

SEQ ID NO: 361 - DNA, 55 bp, Artificial Sequence
  Description of Artificial Sequence Synthetic primer
ccttattttaacttgctatttctagctctaaaacaggcaggcagctttgcagccg

SEQ ID NO: 362 - DNA, 53 bp, Artificial Sequence
  Description of Artificial Sequence Synthetic primer
cttataagttctgtatgagaccactttttcccaagtgcaacaagcagaaaggg

SEQ ID NO: 363 - DNA, 55 bp, Artificial Sequence
  Description of Artificial Sequence Synthetic primer
ccttattttaacttgctatttctagctctaaaaccctttctgcttgttgcacttg

SEQ ID NO: 364 - DNA, 53 bp, Artificial Sequence
  Description of Artificial Sequence Synthetic primer
cttataagttctgtatgagaccactttttcccaaagagccccacttggaaggg

SEQ ID NO: 365 - DNA, 55 bp, Artificial Sequence
  Description of Artificial Sequence Synthetic primer
ccttattttaacttgctatttctagctctaaaacccttccaagtggggctctttg

SEQ ID NO: 366 - DNA, 53 bp, Artificial Sequence
  Description of Artificial Sequence Synthetic primer
cttataagttctgtatgagaccactttttcccgccccacttggaaggcggttg

SEQ ID NO: 367 - DNA, 55 bp, Artificial Sequence
  Description of Artificial Sequence Synthetic primer
ccttattttaacttgctatttctagctctaaaacaaccgccttccaagtggggcg

SEQ ID NO: 368 - DNA, 53 bp, Artificial Sequence
  Description of Artificial Sequence Synthetic primer
cttataagttctgtatgagaccactttttccctccaaaccgccttccaagtgg

SEQ ID NO: 369 - DNA, 55 bp, Artificial Sequence
  Description of Artificial Sequence Synthetic primer
ccttattttaacttgctatttctagctctaaaaccacttggaaggcggtttggag

SEQ ID NO: 370 - DNA, 53 bp, Artificial Sequence
  Description of Artificial Sequence Synthetic primer
cttataagttctgtatgagaccactttttcccatccaaaccgccttccaagtg

SEQ ID NO: 371 - DNA, 55 bp, Artificial Sequence
  Description of Artificial Sequence Synthetic primer
ccttattttaacttgctatttctagctctaaaacacttggaaggcggtttggatg

SEQ ID NO: 372 - DNA, 25 bp, Artificial Sequence
  Description of Artificial Sequence Synthetic primer
tgctttggattggtaggacctgttc

SEQ ID NO: 373 - DNA, 25 bp, Artificial Sequence
  Description of Artificial Sequence Synthetic primer
ggataatgcacaccttccctgagtc

SEQ ID NO: 374 - DNA, 642 bp, Artificial Sequence
  Description of Artificial Sequence Synthetic polynucleotide
tgctttggattggtaggacctgttccttacatcttacctcctagttacatcttttcctaggattcttaaaactagtatggatatgctgagcatacattctttagaaccttttggactgttttggtaaatttcgtagtcgtaggatcagcacaaagcggaacttgacacacttgtggagttttacggctgtacttggtccttctccatccctttgcttccttttcctaaaccaagtcccagacatgtcaggagaatgaattcatttttaatgccagatgagtttggtgtaagatgcatttgtaaagcaaaataaaaagaatccacaaaacacacaaataaaatccaaaccgccttccaagtggggctctttcatgctgctgctgctgctgctgctgctgctgctgctgctgctgctgctgctgctgctgctgctgctgctgctgctgctcctcctcctcctcctccttctcctcctcctcctcctcttctagaccttcttttggagaaatggctttcggaagttttgccaggaaacgtagccctaggcaggcagctttgcagccccctttctgcttgttgcactttctccattcgttcctttgctttttgcaggctctgactcagggaaggtgtgcattatcc